(19) United States
(12) Patent Application Publication
(10) Pub. No.: US 2023/0092174 A1
(43) Pub. Date: Mar. 23, 2023

(54) ACTIVE AGENT-CONTAINING ARTICLES

(71) Applicant: The Procter & Gamble Company, Cincinnati, OH (US)

(72) Inventors: Mark William HAMERSKY, Hamilton, OH (US); Paul Martin LIPIC, West Chester, OH (US); Klaus Juergen VOLLMER, Cincinnati, OH (US); Joseph Michael GAINES, Cincinnati, OH (US); Rajesh Kant MISHRA, Mason, OH (US)

(21) Appl. No.: 17/878,947

(22) Filed: Aug. 2, 2022

Related U.S. Application Data
(60) Provisional application No. 63/330,405, filed on Apr. 13, 2022.
(60) Provisional application No. 63/228,204, filed on Aug. 2, 2021.

(51) Int. Cl. A61K 8/02; A61Q 9/02; A61K 8/41

(52) U.S. Cl. CPC A61K 8/0208; A61Q 9/02; A61K 8/416

(57) ABSTRACT

Active agent-containing fibrous elements and/or articles, and more particularly to water-insoluble fibrous elements and/or water-insoluble articles, for example consumable, single use water-insoluble articles such as shave prep articles including one or more active agents, for example one or more fatty amphiphiles and one or more cationic surfactants, and one or more auxiliary ingredients, for example structurants, fibrous elements containing such active agents and auxiliary ingredients, methods for making such articles and fibrous elements, and methods of using such articles are provided.

### FIELD OF THE INVENTION

The present invention relates to active agent-containing fibrous elements and/or articles, and more particularly to water-insoluble fibrous elements and/or water-insoluble articles, for example consumable, single use water-insoluble articles such as shave prep articles comprising one or more active agents, for example one or more fatty amphiphile active agents, such as fatty alcohols, fatty acids, fatty quaternary ammonium compounds, and one or more cationic surfactants, and one or more auxiliary ingredients, for example structurants, such as polymeric structurants, fibrous elements containing such active agents and auxiliary ingredients, methods for making such articles and fibrous elements, and methods of using such articles.

### BACKGROUND OF THE INVENTION

Water-insoluble articles comprising active agents, such as fatty amphiphiles and cationic surfactants, and optionally an auxiliary ingredient, such as a structurant, are known as described in U.S. Pat. No. 10,975,339. However, such known water-insoluble articles and their water-insoluble fibrous elements forming such known water-insoluble articles hydrate too much for shave prep applications causing such water-insoluble fibrous elements and/or water-insoluble articles to swell too much, for example swell completely and/or fully hydrate even to the point of breaking apart, softening to the point and/or reducing the modulus of the fibrous elements to the point that the fibrous elements lose their fibrous element physical structure, integrity and strength. When the known water-insoluble fibrous elements swell as described above, the water-insoluble fibrous elements and/or water-insoluble article comprising such water-insoluble fibrous elements spread over surfaces, such as skin, when shear is applied to the water-insoluble fibrous elements and/or water-insoluble articles. While such properties and/or characteristics of such known water-insoluble fibrous elements and/or water-insoluble articles may be desirable for hair conditioning applications, such properties and/or characteristics are undesirable for shave prep applications since cause negatives in rinsing off of the shave prep composition, for example cream, after application to skin, and/or negatives in rinsing off of the shave prep composition from a razor after use.

As shown in FIGS. 1 and 2, one problem with such known water-insoluble articles (Comparative Example in FIGS. 1 and 2) and water-insoluble fibrous elements making such water-insoluble articles is that such known water-insoluble articles and/or water-insoluble fibrous elements hydrate too much making them unsuitable for use for certain applications, such as shave prep applications, due to the negative discussed above.

Accordingly, there is a need for water-insoluble articles and/or water-insoluble fibrous elements comprising one or more active agents, such as fatty amphiphiles and cationic surfactants, and optionally an auxiliary ingredient, such as a structurant, that exhibit hydration properties such that they do not swell to much and/or break apart during use and/or exhibit other negatives described above that exist in known water-insoluble articles and/or water-insoluble fibrous elements, methods for making such articles and fibrous elements, and methods of using such articles.

### SUMMARY OF THE INVENTION

The present invention fulfills the needs described above by providing a water-insoluble article, for example a consumable, single use, water-insoluble active agent-containing article, such as a consumable, single use, water-insoluble active agent-containing shave prep article, water-insoluble fibrous elements used in the water-insoluble article, methods for making same, and a methods for using same.

One solution to the problem identified above is to provide an active agent-containing fibrous elements and/or article comprising such fibrous elements, and more particularly to a water-insoluble article, for example a consumable, single use water-insoluble article such as a shave prep article wherein the fibrous elements of the article comprise one or more active agents, for example one or more shave prep active agents and/or one or more fatty amphiphile active agents and one or more cationic surfactants, and one or more auxiliary ingredients, for example structurants, such as polymeric structurants, wherein the one or more active agents and the one or more auxiliary ingredients are present in a plurality of water-insoluble fibrous elements that form the water-insoluble article such that the water-insoluble fibrous elements and water-insoluble article avoids the negatives of too much hydration as shown in FIGS. 1 and 2 (Examples 1 and 3 in FIGS. 1 and 2), methods for making such articles and fibrous elements, and methods of using such articles. Further, the water-insoluble article may form a lamellar structure (exhibit a lamellar structure response) as measured by the Lamellar Structure Test Method described herein.

In one example of the present invention, a water-insoluble fibrous element comprising:

a. one or more active agents;

b. one or more auxiliary ingredients; and

c. a fibrous element hydration controlling system, is provided.

In another example of the present invention, a water-insoluble article comprising a plurality of water-insoluble fibrous elements comprising one or more active agents, one or more auxiliary ingredients, and a fibrous element hydration controlling system, is provided.

In yet another example of the present invention, a water-insoluble article comprising a plurality of water-insoluble fibrous elements comprising one or more active agents, one or more auxiliary ingredients, and a fibrous element hydration controlling system and a plurality of particles, is provided.

In still another example of the present invention, a water-insoluble article comprising a plurality of water-insoluble fibrous elements comprising one or more active agents and one or more auxiliary ingredients, and optionally a fibrous element hydration controlling system, wherein the water-insoluble article further comprises an external fibrous element hydration controlling system, is provided.

In even another example of the present invention, a water-insoluble article comprising a plurality of water-insoluble fibrous elements comprising one or more active agents, one or more auxiliary ingredients, and a fibrous element hydration controlling system, wherein the water-insoluble article exhibits a lamellar structure response as measured according to the Lamellar Structure Test Method, is provided.

In even another example of the present invention, a water-insoluble article comprising a plurality of water-insoluble fibrous elements comprising one or more active agents and one or more auxiliary ingredients, and optionally a fibrous element hydration controlling system, wherein the water-insoluble article exhibits a lamellar structure response as measured according to the Lamellar Structure Test Method, is provided.

In even still another example of the present invention, a water-insoluble article comprising a plurality of water-insoluble fibrous elements comprising one or more active agents, one or more auxiliary ingredients, and a fibrous element hydration controlling system, wherein the water-insoluble article exhibits a lamellar structure response in a wet state but does not exhibit a lamellar structure response in a dry state as measured according to the Lamellar Structure Test Method, is provided.

In even still another example of the present invention, a water-insoluble article comprising a plurality of water-insoluble fibrous elements comprising one or more active agents and one or more auxiliary ingredients, and optionally a fibrous element hydration controlling system, wherein the water-insoluble article exhibits a lamellar structure response in a wet state but does not exhibit a lamellar structure response in a dry state as measured according to the Lamellar Structure Test Method, is provided.

In even yet another example of the present invention, a water-insoluble article comprising a plurality of the water-insoluble fibrous elements of the present invention, wherein the plurality of fibrous elements comprise one or more active agents and one or more auxiliary ingredients, and wherein the water-insoluble article further comprises a plurality of particles, wherein the plurality of particles comprise an external fibrous element hydration controlling system.

In still another example of the present invention, an article, for example a water-insoluble article, comprising a plurality of fibrous elements, for example a plurality of water-insoluble fibrous elements, that when exposed to conditions of intended use, such as contacted with water, such as in a shave prep application to create a cream from the article, swells less than known articles, such as hair conditioning articles, for example known water-insoluble articles, is provided.

In still another example of the present invention, an article, for example a water-insoluble article, comprising a plurality of fibrous elements, for example a plurality of water-insoluble fibrous elements, that when exposed to conditions of intended use, such as contacted with water, such as in a shave prep application to create a cream from the article, wherein the article, for example water-insoluble article, and/or fibrous elements, for example water-insoluble fibrous elements, swell less than 100% and/or less than 80% and/or less than 60% and/or less than 50% and/or less than 40% and/or less than 30% and/or less than 20% and/or less than 10% and/or to about 0% and/or to about 5% their original state prior to being exposed to conditions of intended use, for example being contacted with water, for example after 30 seconds and/or after 1 minute and/or after 5 minutes and/or after 10 minutes and/or after 20 minutes and/or after 30 minutes after being contacted with water and optionally sheared by rubbing, for example in one's hands, is provided.

In still another example of the present invention, a method for making a fibrous element-forming composition comprising the steps of:

a. melting one or more active agents to form a melt;

b. adding one or more auxiliary ingredients to the melt; and

c. adding a fibrous element hydration controlling system to the one or more active agents prior to, concurrently or after melting the one or more active agents such that a fibrous element-forming composition is formed, is provided.

In even yet another example of the present invention, a method for making a plurality of fibrous elements, the method comprising the steps of:

a. providing a fibrous element-forming composition according to the present invention; and

b. producing a plurality of fibrous elements from the fibrous element-forming composition, is provided.

In still yet another example of the present invention, a method for making an article of the present invention comprising the following steps:

a. subjecting one or more active agents to a temperature sufficient to melt the active agents;

b. adding one or more auxiliary ingredients, for example structurants, to the melted active agents to form a fibrous element-forming composition;

c. producing a plurality fibrous elements from the fibrous element-forming composition, for example by spinning the fibrous element-forming composition;

d. adding one or more fibrous element hydration controlling systems to the melted active agents prior to or after adding the one or more auxiliary ingredients and/or adding one or more external fibrous element hydration controlling systems to the plurality of fibrous elements formed from the fibrous element-forming composition;

e. collecting a plurality of the fibrous elements, with or with one or more of the external fibrous element hydration controlling systems on a collection device to form a fibrous structure and/or article comprising the plurality of fibrous elements according to the present invention, is provided.

In even still yet another example of the present invention, a method for using the article of the present invention comprises the steps of:

a. providing an article according to the present invention;

b. adding a liquid, such as water, to the article to form a wet article;

c. transforming the wet article into a cream, for example by rubbing in a user's hands;

d. applying the cream to a user's skin and/or hair; and

e. removing at least a portion of the cream with a razor and/or razor blade during a shaving operation, is provided.

The present invention provides novel fibrous elements, fibrous structures and articles useful in shave prep applications, and methods for making same and methods for using same.

### BRIEF DESCRIPTION OF THE DRAWINGS

FIG. 1 are images of Comparative Example fibrous elements and examples of the present invention, Examples 1 and 3, fibrous elements that illustrate the hydration of such fibrous elements;

FIG. 2 are images of resulting creams produced from the Comparative Example article and an example of the present invention, Example 3, article;

FIG. 3 is a schematic representation of an example of a fibrous element, in this case a filament, according to the present disclosure;

FIG. 4 is a schematic representation of an example of a fibrous structure comprising a plurality of filaments according to the present disclosure;

FIG. 5 is a scanning electron microscope photograph of a cross-sectional view of an example of a fibrous structure according to the present disclosure;

FIG. 6 is a schematic representation of a cross-sectional view of another example of a fibrous structure according to the present disclosure;

FIG. 7 is a schematic representation of a cross-sectional view of another example of a fibrous structure according to the present disclosure;

FIG. 8 is a scanning electron microscope photograph of a cross-sectional view of another example of a fibrous structure according to the present disclosure;

FIG. 9 is a schematic representation of an example of a process for making an example of a fibrous structure according to the present disclosure;

FIG. 10 is a schematic representation of an example of a die with a magnified view used in the process of FIG. 9;

FIG. 11 is a schematic representation of an example of another process for making an example of a fibrous structure according to the present disclosure;

FIG. 12 is a schematic representation of another example of a process for making another example of a fibrous structure according to the present disclosure; and

FIG. 13 is a schematic representation of another example of a process for making another example of a fibrous structure according to the present disclosure.

### DETAILED DESCRIPTION OF THE INVENTION

“Article”, for example a “water-insoluble article” as used herein refers to a consumer use unit, a consumer unit dose unit, a consumer use saleable unit, a single dose unit, or other use form comprising one or more fibrous structures, for example one or more water-insoluble fibrous structures, such as one or more unitary fibrous structures, for example one or more water-insoluble unitary fibrous structures and/or a plurality of water-insoluble fibrous elements of the present invention.

In one example, the article, for example a fibrous structure, of the present invention comprises a plurality of fibrous elements at a basis weight of greater than 1 g/m² and/or greater than 10 g/m² and/or greater than 20 g/m² and/or greater than 30 g/m² and/or greater than 40 g/m² and/or from about 1 g/m² to about 3000 g/m² and/or from about 10 g/m² to about 5000 g/m² and/or to about 3000 g/m² and/or to about 2000 g/m² and/or from about 20 g/m² to about 2000 g/m² and/or from about 30 g/m² to about 1000 g/m² and/or from about 30 g/m² to about 500 g/m² and/or from about 30 g/m² to about 300 g/m² and/or from about 40 g/m² to about 100 g/m² and/or from about 40 g/m² to about 80 g/m² as measured by the Basis Weight Test Method described herein. In one example, the article, for example a fibrous structure, comprises two or more layers wherein fibrous elements and/or film are present in at least one of the layers at a basis weight of from about 1 g/m² to about 500 g/m² as measured according to the Basis Weight Test Method described herein.

“Fibrous structure” and/or “water-insoluble fibrous structure” as used herein means a structure that comprises a plurality of fibrous elements, for example a plurality of water-insoluble fibrous elements, and optionally, one or more particles. In one example, a fibrous structure according to the present invention means an association of water-insoluble fibrous elements, in one example inter-entangled, water-insoluble fibrous elements, and optionally, particles that together form a structure, such as a unitary structure, capable of performing a function, such as a shave prep function. Particles may be blended with the water-insoluble fibrous elements, for example 1) in a coform state via a coform process, 2) in a layered state between layers or plies of the water-insoluble fibrous elements, and/or 3) combinations of these two in a fibrous structure of the present invention and/or article of the present invention.

The fibrous structures of the present invention may be homogeneous or may be layered. If layered, the fibrous structures may comprise at least two and/or at least three and/or at least four and/or at least five layers, for example one or more fibrous element layers, one or more particle layers and/or one or more fibrous element/particle mixture layers. A layer may comprise a particle layer within the fibrous structures or between fibrous element layers in a fibrous structure.

In one example, one or more fibrous structures of the present invention may be associated, for example by bonding, such as thermal or adhesive or pressure bonding, with one or more other fibrous structures to form a multi-ply fibrous structure.

In one example, a fibrous structure or fibrous structure ply, for example a single-ply fibrous structure, according to the present invention or a multi-ply fibrous structure comprising one or more fibrous structure plies according to the present invention may exhibit a basis weight of less than 5000 g/m² as measured according to the Basis Weight Test Method described herein. In one example, the single- or multi-ply fibrous structures according to the present invention may exhibit a basis weight of greater than 10 g/m² to about 5000 g/m² and/or greater than 10 g/m² to about 3000 g/m² and/or greater than 10 g/m² to about 2000 g/m² and/or greater than 10 g/m² to about 1000 g/m² and/or greater than 20 g/m² to about 800 g/m² and/or greater than 30 g/m² to about 600 g/m² and/or greater than 50 g/m² to about 500 g/m² and/or greater than 300 g/m² to about 3000 g/m² and/or greater than 500 g/m² to about 2000 g/m² as measured according to the Basis Weight Test Method.

In one example, the fibrous structure of the present invention is a “unitary fibrous structure.”

“Unitary fibrous structure” as used herein is an arrangement comprising one or more particles and a plurality of two or more and/or three or more fibrous elements that are inter-entangled or otherwise associated with one another to form a fibrous structure. A unitary fibrous structure of the present invention may be one or more plies within a multi-ply fibrous structure. In one example, a unitary fibrous structure of the present invention may comprise three or more different fibrous elements. In another example, a unitary fibrous structure of the present invention may comprise two different fibrous elements, for example a co-formed fibrous structure, upon which a different fibrous element is deposited to form a fibrous structure comprising three or more different fibrous elements.

“Fibrous element” as used herein means an elongate particulate having a length greatly exceeding its average diameter, i.e. a length to average diameter ratio of at least about 10. A fibrous element may be a filament or a fiber. In one example, the fibrous element is a single fibrous element rather than a yarn comprising a plurality of fibrous elements.

The fibrous elements of the present invention may be spun from fibrous element-forming compositions also referred to as filament-forming compositions via suitable spinning process operations, such as meltblowing, spunbonding, electro-spinning, and/or rotary spinning.

The fibrous elements of the present invention may be monocomponent (single, unitary solid piece rather than two different parts, like a core/sheath bicomponent) and/or multicomponent. For example, the fibrous elements may comprise bicomponent fibers and/or filaments. The bicomponent fibers and/or filaments may be in any form, such as side-by-side, core and sheath, islands-in-the-sea and the like.

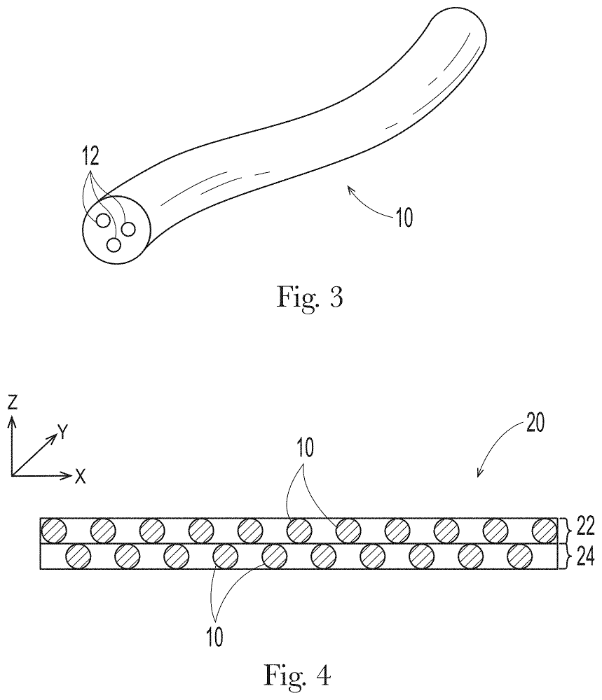

In one example as shown in FIG. 3, a fibrous element 10, for example a filament, of the present invention made from a fibrous element-forming composition of the present invention is such that one or more active agents 12 may be present in the fibrous element 10 rather than on a surface of the fibrous element, such as a coating composition comprising one or more active agents, which may be the same or different from the active agents in the fibrous elements and/or particles, if present. The total level of fibrous element-forming materials and total level of active agents present in the fibrous element-forming composition may be any suitable amount so long as the fibrous elements of the present invention are produced therefrom.

In one example, one or more active agents, may be present in the fibrous elements and one or more additional active agents, for example optional active agents, may be present on a surface of the fibrous elements as a coating or partial coating. In another example, a fibrous element of the present invention may comprise one or more active agents that are present in the fibrous element when originally made, but then bloom to a surface of the fibrous element prior to and/or when exposed to conditions of intended use of the fibrous element.

“Filament” as used herein means an elongate particulate as described above that exhibits a length of greater than or equal to 5.08 cm (2 in.) and/or greater than or equal to 7.62 cm (3 in.) and/or greater than or equal to 10.16 cm (4 in.) and/or greater than or equal to 15.24 cm (6 in.).

Filaments are typically considered continuous or substantially continuous in nature. Filaments are relatively longer than fibers.

“Fiber” as used herein means an elongate particulate as described above that exhibits a length of less than 5.08 cm (2 in.) and/or less than 3.81 cm (1.5 in.) and/or less than 2.54 cm (1 in.).

Fibers are typically considered discontinuous in nature. Non-limiting examples of fibers include staple fibers produced by spinning a filament or filament tow of the present invention and then cutting the filament or filament tow into segments of less than 5.08 cm (2 in.) thus producing fibers.

In one example, one or more fibers may be formed from a filament of the present invention, such as when the filaments are cut to shorter lengths (such as less than 5.08 cm in length). Thus, in one example, the present invention also includes a fiber made from a filament of the present invention, such as a fiber comprising one or more filament-forming materials and one or more active agents. Therefore, references to filament and/or filaments of the present invention herein also include fibers made from such filament and/or filaments unless otherwise noted. Fibers are typically considered discontinuous in nature relative to filaments, which are considered continuous in nature.

“Fibrous element-forming composition” and/or “filament-forming composition” as used herein means a non-aqueous composition that is suitable for making a fibrous element of the present invention such as by spinning, for example meltblowing or spunbonding (in the case of fibrous elements). The fibrous element-forming composition comprises one or more active agents suitable for producing a fibrous element and/or plurality of fibrous elements, such as by spinning into a plurality of fibrous elements. In addition to the one or more active agents, the fibrous element-forming composition may comprise one or more auxiliary ingredients, such as one or more filament-forming materials, for example one or more structurants, that exhibit properties that make the fibrous element-forming composition more suitable for spinning into a fibrous element. In one example, the auxiliary ingredients comprise one or more structurants, such as one or more polymer structurants and/or inorganic structurants. In one example, the one or more auxiliary ingredients are present in the fibrous element-forming composition and the resulting fibrous elements formed therefrom at less than 50% and/or less than about 45% and/or less than about 40% and/or less than about 35% and/or less than about 30% and/or less than about 25% and/or to greater than 0% and/or to greater than about 5% and/or to greater than about 10% and/or to greater than about 15% and/or from about 1% to less than 50% by weight of the fibrous element-forming composition and the resulting fibrous elements formed therefrom. In one example, the one or more active agents are present in the fibrous element-forming composition and the resulting fibrous elements formed therefrom at greater than about 20% and/or greater than about 30% and/or greater than about 40% and/or greater than about 50% and/or greater than about 60% and/or greater than about 70% to about 99% and/or to about 95% and/or to about 90% and/or to about 85% and/or to about 80% by weight of the fibrous element-forming composition and fibrous elements formed therefrom.

In one example, the filament-forming composition may be made by heating and optionally stirring one or more active agents until the melted active agents are homogeneous. Then the homogeneous melted active agents, which in this case is the fibrous element-forming composition, can be spun into a plurality of fibrous elements. Alternatively, one or more auxiliary ingredients, such as fibrous element-forming materials, for example structurants, such as polymeric structurants and/or inorganic structurants, may be added, with or without stirring and/or agitation, to the homogeneous melted active agents and dissolved, for example homogeneously dissolved, in and/or dispersed, for example homogeneously dispersed, in the one or more active agents, for example one or more melted active agents to form the fibrous element-forming composition, which can then be spun into a plurality of fibrous elements.

“Active agent” as used herein means a material that produces an intended effect in an environment external to an article of the present invention, such as when the article is exposed to conditions of intended use of the article. In one example, an active agent comprises a material that treats a surface, such as a soft surface (i.e., hair, skin) during use of the article by a user for shave prep purposes.

“Treats” as used herein with respect to treating a surface means that the active agent provides a benefit to a surface or environment. Treats includes regulating and/or immediately improving a surface's or environment's appearance, cleanliness, smell, purity and/or feel and/or suppleness and/or smoothness. In one example, treating in reference to treating a keratinous tissue (for example skin and/or hair) surface means regulating and/or immediately improving the keratinous tissue's cosmetic appearance and/or feel. For instance, “regulating skin or hair (keratinous tissue) condition” includes: thickening of skin or hair (e.g, building the epidermis and/or dermis and/or sub-dermal [e.g., subcutaneous fat or muscle] layers of the skin, and where applicable the keratinous layers of the nail and hair shaft) to reduce skin or hair atrophy, increasing the convolution of the dermal-epidermal border (also known as the rete ridges), preventing loss of skin or hair elasticity (loss, damage and/or inactivation of functional skin elastin) such as elastosis, sagging, loss of skin or hair recoil from deformation; melanin or non-melanin change in coloration to the skin or hair, such as under eye circles, blotching (e.g., uneven red coloration due to, e.g., rosacea) (hereinafter referred to as “red blotchiness”), sallowness (pale color), discoloration caused by telangiectasia or spider vessels, and graying hair.

“Shave prep active agent” as used herein means an active agent that may be useful for treating keratinous tissue (e.g., hair or skin) condition especially during a shave prep experience.

“Auxiliary ingredient” and/or “fibrous element-forming material” and/or “filament-forming material” as used herein means a material, such as a structurant, for example a polymeric structurant and/or an inorganic structurant, that exhibits properties within the fibrous element-forming composition that aid the making a fibrous element, such as by spinning the fibrous element-forming composition. Also, inorganic structurants can act as fillers, viscosity modifiers, and/or to build the fibrous elements. In one example, the auxiliary ingredient is a structurant. A “structurant” as used herein means a material, for example a polymer, such as a polymeric structurant, that improves the fibrous element spinning of the melted active agents, such as the fatty amphiphiles, such as fatty alcohols, fatty quaternary ammonium compounds, fatty acids, etc. The structurant increases the shear and extensional viscosity of the melted active agents to enable fibrous element formation. In one example, the structurant exhibits a weight average molecular weight of from about 10,000 to about 4,000,000 g/mol and/or from about 15,000 to about 3,000,000 g/mol and/or from about 20,000 to about 2,500,000 g/mol and/or from about 30,000 to less than 2,000,000 g/mol and/or from about 40,000 to about 1,700,000 g/mol. The weight average molecular weight is computed by summing the average molecular weights of each polymer raw material multiplied by their respective relative weight percentages by weight of the total weight of polymers present within the article. In one example, the structurant is soluble in an oily mixture to enable viscosity build for fibrous element spinning. In addition, the structurant may also be soluble in water to promote removal and to prevent buildup. Suitable structurants include, but are not limited to, polyvinylpyrrolidone, polydimethylacrylamides, and combinations thereof. These polymers are oil (fatty alcohol, fatty acid, fatty quaternary ammonium compounds) soluble, water soluble, water miscible, and capable of being produced at high molecular weights. For example, suitable polymers for use are PVP K90, a polyvinylpyrrolidone from Ashland Inc., having a molecular weight of about 1,000,000 to about 1,700,000 g/mol and PVP K30, a polyvinylpyrrolidone from Ashland Inc., having a molecular weight of about 40,000 to about 80,000 g/mol, which enable fibrous elements to be spun, formed and collected onto a collection device, such as a belt. Additional suitable polymers include copolymers of polyvinylpyrrolidone, such as Ganex® or PVP/VA (weight average molecular weight of about 50,000 g/mol) copolymers from Ashland Inc., which also function as suitable structurants but require a higher level to be effective due to their lower molecular weights. In addition, copolymers of polydimethylacrylamide also function as suitable structurants. Hydroxyl propyl cellulose can also function as a suitable structurant.

Non-limiting examples of structurants suitable for the present invention include polymeric structurants, inorganic structurants, and mixtures thereof. In one example, the structurant comprises a polymeric structurant selected from the group consisting of: polylactams such as polyvinylpyrrolidone and copolymers of vinylpyrrolidone, polydimethylacrylamide, copolymers of dimethylacrylamide, and mixtures thereof. In one example, the structurant comprises polyvinylpyrrolidone. In another example, the structurant comprises polydimethylacrylamide. In still another example, the structure comprises polyvinylpyrrolidone and polydimethylacrylamide. In one example, the structurant comprises inorganic structurants selected from the group consisting of clays, silica, and mixtures thereof.

In one example, the auxiliary ingredient, for example structurant comprises one or more substituted polymers such as a cationic polymer.

As used herein, “vinyl pyrrolidone copolymer” (and “copolymer” when used in reference thereto) refers to a polymeric structurant of the following structure:

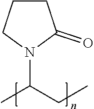

wherein n is an integer such that the polymeric structurant has the degree of polymerization such that it possesses characteristics described herein. For purposes of clarity, the use of the term “copolymer” is intended to convey that the vinyl pyrrolidone monomer can be copolymerized with other non-limiting monomers such as vinyl acetate, alkylated vinyl pyrrolidone, vinyl caprolactam, acrylic acid, methacrylate, acrylamide, methacrylamide, dimethacrylamide, alkylaminomethacrylate, and alkylaminomethacrylamide monomers.

“Fibrous element hydration controlling system” as used herein means a material or combination of materials, which may function both as a fibrous element hydration controlling material and as an active agent, that prevent and/or inhibit and/or delay and/or reduce the hydration of one or more of the fibrous elements, for example one or more of the water-insoluble fibrous elements, such as a plurality of water-insoluble fibrous elements of the present invention, when such fibrous elements are contacted with water, such as excess water from a tap, during use of the fibrous elements and/or an article, such as a water-insoluble article, for example a shave prep water-insoluble article comprising the fibrous elements. As shown in FIG. 1 Comparative Example, fibrous elements that lack a fibrous element hydration controlling system exhibit an undesirable level of hydration of the fibrous elements, results in the fibrous elements and/or article comprising the fibrous elements swelling too much causing negative performance, including the fibrous elements swelling completely and/or losing their integrity by breaking apart, which results in the fibrous elements spreading too easily upon shearing and/or exhibiting rinsing negatives, for example from the user's skin and/or user's razor. In one example, the fibrous element hydration controlling system comprises one or more hydration controlling materials that are present in the fibrous elements and/or a fibrous element-forming composition from which the fibrous elements are produced. In one example, the fibrous element hydration controlling system results in the fibrous element and/or the plurality of fibrous elements and/or the article comprising the fibrous elements from hydrating

“External fibrous element hydration controlling system” as used herein means a material or combination of materials, which may function both as an external fibrous element hydration controlling material and as an active agent, that prevent and/or inhibit and/or delay and/or reduce the hydration of one or more of the fibrous elements, for example one or more of the water-insoluble fibrous elements, such as a plurality of water-insoluble fibrous elements of the present invention, when such fibrous elements are contacted with water, such as excess water from a tap, during use of the fibrous elements and/or an article, such as a water-insoluble article, for example a shave prep water-insoluble article comprising the fibrous elements. In one example, the external fibrous element hydration controlling system comprises one or more hydration controlling materials that are present external to the fibrous elements and/or a fibrous element-forming composition from which the fibrous elements are produced. In one example, the external fibrous element hydration controlling system may be present on an external surface of the fibrous elements, for example as a coating. In another example, the external fibrous element hydration controlling system may be discrete and separate from the fibrous elements and/or may exist as a mixture of discrete fibrous elements and a discrete external fibrous element hydration controlling system in an article of the present invention. In one example, the external fibrous element hydration controlling system comprises a salt-generating system. In another example, the external fibrous element hydration controlling system comprises an effervescent system.

“Salt-generating system” as used herein, for example as a fibrous element hydration controlling system and/or external fibrous element hydration controlling system, means a material and/or combination of materials that create a salt upon being exposed to a liquid, such as water, for example during use of the fibrous elements and/or article of the present invention for shave prep purposes.

“Effervescent system” as used herein means a material or combination of materials that generates effervescence, for example gas, such as CO₂. In one example, the effervescent system comprises an effervescent acid or effervescent acid particle and an effervescent salt or effervescent salt particle. In one example, the effervescent system comprises an agglomerate comprising an effervescent acid or effervescent acid particle and an effervescent salt or effervescent salt particle.

In one example, the selection of specific effervescent acids, for example effervescent acid particles, and/or effervescent salts, for example effervescent salt particles, and their proportions depends, at least in part, upon the requirements for the amount of gas, for example CO₂ release. In one example, the effervescent acid, for example effervescent acid particle, such as citric acid, may be added in an amount of about 10% to about 60% by weight of the effervescent system, while the effervescent salt, for example effervescent salt particle, such as an alkali metal salt, for example sodium bicarbonate, may also be added in an amount of about 10% to 60% by weight of the effervescent components.

“Effervescent acid” or “Effervescent acid particle” as used herein means an acid and/or acid particle that generates effervescence, for example gas, such as CO₂, when combined with an Effervescent salt or Effervescent salt particle. Non-limiting examples of suitable effervescent acids and/or effervescent acid particles for use in the foaming compositions of the present invention include, but are not limited to, tartaric acid, citric acid, fumaric acid, adipic acid, malic acid, oxalic acid, sulfamic acid, and mixtures thereof. In one example, the effervescent acid and/or effervescent acid particle comprises citric acid or a mixture of citric acid and tartaric acid. The effervescent acid and/or effervescent acid particle may be anhydrous.

“Effervescent salt” or “Effervescent salt particle” as used herein means a salt and/or salt particle that generates effervescence, for example gas, such as CO₂, when combined with an effervescent acid and/or effervescent acid particle. Non-limiting examples of suitable effervescent salts and/or effervescent salt particles include, but are not limited to, alkali metal salts and/or carbonate salts and/or bicarbonate salts, such as sodium carbonate, calcium carbonate, magnesium carbonate, ammonium carbonate, potassium carbonate, sodium bicarbonate, calcium bicarbonate, and mixtures thereof. The effervescent salt and/or effervescent salt particle may be anhydrous.

“Particle”, for example “effervescent acid particle” and/or “effervescent salt particle” and/or “agglomerate” and/or “active agent-containing particle” and/or “optional active agent-containing particle” as used herein means a powder, granule, encapsulate, microcapsule, matrix particle, prill and/or agglomerate. The shape of the particle can be in the form of spheres, rods, plates, tubes, squares, rectangles, discs, stars, fibers or have regular or irregular random forms. The particles of the present invention, at least those of at least 44 μm, can be measured by the Median Particle Size Test Method described herein. For particles that are less than 44 μm, a different test method may be used, for example light scattering, to determine the particle sizes less than 44 μm, for example perfume microcapsules that typically range from about 15 μm to about 44 μm and/or about 25 μm in size. In one example, the particle exhibits a median particle size of 2000 μm or less as measured according to the Median Particle Size Test Method described herein. In another example, the particle exhibits a median particle size of from about 1 μm to about 2000 μm and/or from about 1 μm to about 1600 μm and/or from about 1 μm to about 800 μm and/or from about 5 μm to about 500 μm and/or from about 10 μm to about 300 μm and/or from about 10 μm to about 100 μm and/or from about 10 μm to about 50 μm and/or from about 10 μm to about 30 μm as measured according to the Median Particle Size Test Method described herein.

In one example, the particle of the present invention may exhibit a particle ionic content of less 10 and/or less than 8 and/or less than 6 and/or less than 4 and/or less than 2 μS/g/40 mL of water as measured using a conductivity probe. Non-limiting examples of particles that exhibit a particle ionic content of less than 10 μS/g/40 mL of water are matrix particles, such as aminosilicone and/or a perfume with a hydroxyl polymer, such as polyvinyl alcohol and/or starch, as a carrier. Non-limiting examples of matrix particles suitable for inclusion in the article of the present invention are described in U.S. Patent Application Publication No. 20200093711 A1, which is incorporated by reference.

In one aspect, particles may comprise recycled fibrous structure materials, specifically where said fibrous materials are recycled by grinding fibers into a finely-divided solid and re-incorporating said finely-divided solids into agglomerates, granules or other particle forms. In another aspect, particles may comprise re-cycled fibrous-structure materials, specifically where said fibrous materials are incorporated into a fluid paste, suspension or solution, and then processed to form agglomerates, granules or other particle forms. In another aspect, said fluid pastes, suspensions or solutions comprising recycled fibrous materials may be directly applied to fibrous layers in the process of making new fibrous articles.

In one example, the particles of the present invention, for example the effervescent acid particles and/or effervescent salt particles, which may be coated particles, for example coated effervescent salt particles, exhibit a D50 of less than 500 μm and/or less than 450 μm and/or less than 400 μm and/or less than 350 μm to about 100 μm and/or to about 150 μm and/or to about 200 μm as measured according to the Median Particle Size Test Method described herein, which better provide a stable foam compared to such particles that exhibit a D50 of greater than 1000 μm as measured according to the Median Particle Size Test Method described herein.

In one example, the particles, which may be discrete particles and/or agglomerates (discrete particles bound together, for example by a surfactant) may exhibit a D50 particle size of from about 100 μm to about 5000 μm and/or from about 100 μm to about 2000 μm and/or from about 250 μm to about 1200 μm and/or from about 250 μm to about 850 μm as measured according to the Median Particle Size Test Method described herein.

In one example, the particles, which may be discrete particles and/or agglomerates (discrete particles bound together, for example by a surfactant), may exhibit a D10 of 250 μm as measured according to the Median Particle Size Test Method described herein.

In another example, the particles, which may be discrete particles and/or agglomerates (discrete particles bound together, for example by a surfactant), may exhibit a D90 of 1200 μm and/or 850 μm as measured according to the Median Particle Size Test Method described herein.

In one example, the particles, which may be discrete particles and/or agglomerates (discrete particles bound together, for example by a surfactant), may exhibit a D10 of greater than 44 μm and/or greater than 90 μm and/or greater than 150 μm and/or greater than 212 μm and/or greater than 300 μm as measured according to the Median Particle Size Test Method described herein.

In one example, the particles, which may be discrete particles and/or agglomerates (discrete particles bound together, for example by a surfactant), may exhibit a D90 of less than 1400 μm and/or less than 1180 μm and/or less than 850 μm and/or less than 600 μm and/or less than 425 μm as measured according to the Median Particle Size Test Method described herein. In one example, the particles, which may be discrete particles and/or agglomerates (discrete particles bound together, for example by a surfactant), may exhibit any combination of the above-identified D10, D50, and/or D90 so long as D50, when present, is greater than D10, when present, and D90, when present, is greater than D10 and D50, when present.

In one example, the particles, which may be discrete particles and/or agglomerates (discrete particles bound together, for example by a surfactant), may exhibit any combination of the above-identified D10 and D90 so long as D90 is greater than D10.

In one example, the particles, which may be discrete particles and/or agglomerates (discrete particles bound together, for example by a surfactant), may exhibit a D10 of greater than 212 μm and a D90 of less than 1180 μm as measured according to the Median Particle Size Test Method described herein.

In one example, the particles, which may be discrete particles and/or agglomerates (discrete particles bound together, for example by a surfactant), may exhibit a D10 of greater than 90 μm and a D90 of less than 425 μm as measured according to the Median Particle Size Test Method described herein.

“Active agent-containing particle” and/or “optional active agent-containing particle” as used herein means a particle comprising one or more active agents and/or optional active agents. In one example, the active agent-containing particle is an active agent in the form of a particle (in other words, the particle comprises 100% active agent(s)). The active agent-containing particle may exhibit a median particle size of 2000 μm or less as measured according to the Median Particle Size Test Method described herein. In another example, the active agent-containing particle exhibits a median particle size of from about 1 μm to about 2000 μm and/or from about 1 μm to about 800 μm and/or from about 5 μm to about 500 μm and/or from about 10 μm to about 300 μm and/or from about 10 μm to about 100 μm and/or from about 10 μm to about 50 μm and/or from about 10 μm to about 30 μm as measured according to the Median Particle Size Test Method described herein. In one example, one or more of the active agents is in the form of a particle that exhibits a median particle size of 20 μm or less as measured according to the Median Particle Size Test Method described herein.

In one example of the present invention, the article, for example fibrous structure, comprises a plurality of particles, for example active agent-containing particles and/or optional active agent-containing particles, and a plurality of fibrous elements in a weight ratio of particles, for example active agent-containing particles and/or optional active agent-containing particles to fibrous elements of 1:100 or greater and/or 1:50 or greater and/or 1:10 or greater and/or 1:3 or greater and/or 1:2 or greater and/or 1:1 or greater and/or 2:1 or greater and/or 3:1 or greater and/or 4:1 or greater and/or 5:1 or greater and/or 7:1 or greater and/or 8:1 or greater and/or 10:1 or greater and/or from about 10:1 to about 1:100 and/or from about 8:1 to about 1:50 and/or from about 7:1 to about 1:10 and/or from about 7:1 to about 1:3 and/or from about 6:1 to 1:2 and/or from about 5:1 to about 1:1 and/or from about 4:1 to about 1:1 and/or from about 3:1 to about 1.5:1.

In another example of the present invention, the article, for example a fibrous structure, comprises a plurality of particles, for example active agent-containing particles and/or optional active agent-containing particles, and a plurality of fibrous elements in a weight ratio of particles, for example active agent-containing particles and/or optional active agent-containing particles, to fibrous elements of from about 10:1 to about 1:1 and/or from about 8:1 to about 1.5:1 and/or from about 7:1 to about 2:1 and/or from about 6:1 to about 2.5:1.

In yet another example of the present invention, the article, for example a fibrous structure, comprises a plurality of particles, for example active agent-containing particles and/or optional active agent-containing particles, and a plurality of fibrous elements in a weight ratio of particles, for example active agent-containing particles and/or optional active agent-containing particles, to fibrous elements of from about 1:1 to about 1:100 and/or from about 1:15 to about 1:80, and/or from about 1:2 to about 1:60 and/or from about 1:3 to about 1:50 and/or from about 1:3 to about 1:40.

In another example, the article, for example a fibrous structure, of the present invention comprises a plurality of particles, for example active agent-containing particles and/or optional active agent-containing particles, at a basis weight of greater than 1 g/m² and/or greater than 10 g/m² and/or greater than 20 g/m² and/or greater than 30 g/m² and/or greater than 40 g/m² and/or from about 1 g/m² to about 5000 g/m² and/or to about 3500 g/m² and/or to about 2000 g/m² and/or from about 1 g/m² to about 2000 g/m² and/or from about 10 g/m² to about 1000 g/m² and/or from about 10 g/m² to about 500 g/m² and/or from about 20 g/m² to about 400 g/m² and/or from about 30 g/m² to about 300 g/m² and/or from about 40 g/m² to about 200 g/m² as measured by the Basis Weight Test Method described herein.

In another example, the article, for example a fibrous structure, such as a water-insoluble article, of the present invention comprises a plurality of fibrous elements comprising one or more auxiliary ingredients, for example one or more structurants, such as one or more polymer structurants and/or inorganic structurants, such that the one or more auxiliary ingredients are present in the fibrous element-forming composition and the resulting fibrous elements formed therefrom at a level of greater than 0% to 100% and/or greater than 10% to less than 95% and/or by weight by weight of the fibrous element-forming composition and the resulting fibrous elements formed therefrom, and the article further comprises a plurality of particles, for example active agent-containing particles and/or optional active agent-containing particles. by weight of the fibrous element-forming composition and the resulting fibrous elements formed therefrom.

“Commingled” and/or “commingling” as used herein means the state or form where particles are mixed with fibrous elements, for example filaments. The mixture of fibrous elements and particles can be throughout a composite structure or within a plane or a region of the composite structure. In one example, the commingled fibrous elements and particles may form at least a surface of a composite structure. In one example, the particles may be homogeneously dispersed in the composite structure and/or plane and/or region of the composite structure. In one example, the particles may be homogeneously distributed in the composite structure, which avoids and/or prevents sag and/or free movement and/or migration of the particles within the composite structure to other areas within the composite structure thus resulting in higher concentrated zones of particles and lower concentrated zones or zero concentration zones of particles within the composite structure. In one example, μCT cross-sections of a composite structure can show whether the particles are homogeneously distributed in a composite structure.

“Conditions of intended use” as used herein means the temperature, physical, chemical, and/or mechanical conditions that an article of the present invention is exposed to when the article is used for one or more of its designed purposes. For example, if an article of the present invention is designed to be used as a shave prep product, the conditions of intended use will include those temperature, chemical, physical, including shearing, rubbing, applying to the skin, such as the face, and/or mechanical conditions present during shave prepping and/or shaving wherein the water-insoluble article may form a lamellar structure (exhibit a lamellar structure response) as measured by the Lamellar Structure Test Method described herein.

“Weight ratio” as used herein means the ratio between two materials on their dry basis. For example, the weight ratio of active agents to auxiliary ingredients, such as structurants, within a fibrous element and/or a plurality of fibrous elements is the ratio of the weight of active agents on a dry weight basis (g or %) in the fibrous element to the weight of auxiliary ingredients on a dry weight basis (g or %—same units as the active agents weight) in the fibrous element. In another example, the weight ratio of particles to fibrous elements within a fibrous structure is the ratio of the weight of particles on a dry weight basis (g or %) in the fibrous structure to the weight of fibrous elements on a dry weight basis (g or %—same units as the particle weight) in the fibrous structure.

“Water-insoluble” with respect to an article and/or material as used herein means a fibrous element and/or an article and/or material of the present invention that does not dissolve in excess water and/or is not miscible in water. In other words, a water-insoluble fibrous element and/or article when subjected to agitation in excess water may swell, but not significantly in the presence of a hydration controlling system, but remain intact, and even if it breaks apart into pieces the pieces remain intact in the water and don't dissolve. In one example, a fibrous element and/or article and/or film and/or material that exhibits a lamellar structure (exhibit a lamellar structure response) as determined according to the Lamellar Structure Test Method is considered water-insoluble for purposes of the present invention.

In one example, the fibrous elements and/or article of the present invention is water-insoluble. As defined herein, water-insoluble means that the fibrous elements and/or article do not completely dissolve or disintegrate when exposed to water, for example during a shave prep experience. In one example, when the fibrous elements and/or articles of the present invention are designed for use in shave prep, water-soluble auxiliary ingredients, when present, are used instead of water-insoluble auxiliary ingredients because water-soluble auxiliary ingredients.

“Ambient conditions” as used herein means 23° C.±1.0° C. and a relative humidity of 50%±2%.

“Weight average molecular weight” as used herein means the weight average molecular weight as determined using the industry standard method, gel permeation chromatography.

“Diameter” as used herein, with respect to a fibrous element, is measured according to the Diameter Test Method described herein. In one example, a fibrous element of the present invention exhibits a diameter of less than 100 μm and/or less than 75 μm and/or less than 50 μm and/or less than 25 μm and/or less than 20 μm and/or less than 15 μm and/or less than 10 μm and/or less than 6 μm and/or greater than 1 μm and/or greater than 3 μm as measured according to the Diameter Test Method described herein.

“Triggering condition” as used herein in one example means anything, as an act or event, that serves as a stimulus and initiates or precipitates a change in the article or portion of the article of the present invention, such as a loss or altering of the article's physical structure and/or a release of an active agent therefrom. In another example, the triggering condition may be present in an environment, such as water, heat, shearing during wetting and/or rubbing/shearing of the article for shave prep, wherein the water-insoluble article may form a lamellar structure (exhibit a lamellar structure response) as measured by the Lamellar Structure Test Method described herein that can be applied to the skin, for example face prior to shaving, for example with a razor.

### Article

The articles of the present invention may comprise a plurality of fibrous elements, for example a plurality of filaments, such as a plurality of active agent-containing fibrous elements, and optionally, one or more particles, for example one or more active agent-containing particles and/or optional active agent-containing particles, such as water-soluble, active agent-containing particles and/or water-insoluble particles, such as zeolites, porous zeolites, perfume-loaded zeolites, active-loaded zeolites, silicas, perfume-loaded silicas, active-loaded zeolites, perfume microcapsules, clays, and mixtures thereof.

The article may be in the form of a roll (roll form or rolled product), for example multiple articles connected to adjacent sheets by perforation lines for dispensing individual articles from the rolled form wherein the article is convolutely wound upon itself about a core or without a core to form a rolled article. In one example, a multi-article sheet product comprising a plurality of articles separated from adjacent articles by perforation lines is provided. Alternatively, the article may be in the form of discrete, individual sheets or in a non-rolled form of multiple articles connected to adjacent sheets by perforation lines for dispensing individual articles from the non-rolled form. In yet another example, the article of the present invention is a standalone entity ready for use and a collection and/or number of these entities may be distributed to consumers in a product-shipping assembly, for example a protective product-shipping assembly and/or a container or dispenser for dispensing one or more individual articles.

With respect to an article including one or more fibrous elements, the fibrous elements and/or fibrous structures of the present invention are in solid form. However, the filament-forming composition used to make the fibrous elements of the present invention may be in the form of a liquid.

In one example, the fibrous structure comprises a plurality of identical or substantially identical from a compositional perspective of fibrous elements according to the present invention. In another example, the fibrous structure may comprise two or more different fibrous elements according to the present invention. Non-limiting examples of differences in the fibrous elements may be physical differences such as differences in diameter, length, texture, shape, rigidness, elasticity, and the like; chemical differences such as crosslinking level, solubility, melting point, Tg, active agent, filament-forming material, color, level of active agent, basis weight, level of filament-forming material, presence of any coating on fibrous element, biodegradable or not, hydrophobic or not, contact angle, and the like; differences in whether the fibrous element loses its physical structure when the fibrous element is exposed to conditions of intended use; differences in whether the fibrous element's morphology changes when the fibrous element is exposed to conditions of intended use; and differences in rate at which the fibrous element releases one or more of its active agents when the fibrous element is exposed to conditions of intended use. In one example, two or more fibrous elements and/or particles within the fibrous structure may comprise different active agents. This may be the case where the different active agents may be incompatible with one another, for example an anionic surfactant and a cationic surfactant.

In another example, the fibrous structure may exhibit different regions, such as different regions of basis weight, density and/or caliper. In yet another example, the fibrous structure may comprise texture on one or more of its surfaces. A surface of the fibrous structure may comprise a pattern, such as a non-random, repeating pattern. The fibrous structure may be embossed with an emboss pattern. In another example, the fibrous structure may comprise apertures. The apertures may be arranged in a non-random, repeating pattern.

In one example, the fibrous structure may comprise discrete regions of fibrous elements that differ from other parts of the fibrous structure.

The fibrous structure of the present invention may be used as is or may be coated with one or more active agents.

In one example, a fibrous structure can exhibit a thickness of greater than 0.01 mm and/or greater than 0.05 mm and/or greater than 0.1 mm and/or to about 50 mm and/or to about 20 mm and/or to about 10 mm and/or to about 5 mm and/or to about 2 mm and/or to about 0.5 mm and/or to about 0.3 mm Non-limiting examples of other fibrous structures suitable for the present invention are disclosed in U.S. Published Patent Application No. 2013/0171421 A1 and U.S. Pat. No. 9,139,802 are hereby incorporated by reference herein.

The articles of the present invention may exhibit one or more of the following properties.

In one example, the articles and/or fibrous elements of the present invention may exhibit a lamellar structure (exhibit a lamellar structure response) upon wetting as determined by the Lamellar Structure Test Method described herein.

In one example, the articles and/or fibrous elements of the present invention may exhibit a lamellar structure (exhibit a lamellar structure response) upon wetting as determined by the Lamellar Structure Test Method described herein, but does not exhibit a lamellar structure (exhibit a lamellar structure response) in a conditioned only, dry state as determined by the Lamellar Structure Test Method.

In one example, the article is a non-woven comprising a fibrous structure comprising one or more fibrous elements, for example a plurality of filaments. The article may comprise two or more nonwovens, a multi-ply nonwoven and/or multi-ply fibrous structure.

In one example, the article, for example fibrous structure, may comprise one or more apertures.

In one example, the article, for example fibrous structure, may comprise a graphic printed on one or more surfaces. The graphic may be formed from ink and may traverse the inter-entangled filaments and/or void areas of the surface(s). In one example, the graphic is formed from an ink at least a portion of which penetrates the inter-entangled fibrous elements, for example filaments, and void areas of a surface of the fibrous structure to a depth of 100 microns or less

In one example, the article exhibits a geometric mean peak elongation of about 5% or greater as measured according to the Tensile Test Method.

In one example, the article exhibits a geometric mean modulus of about 5000 g/cm or less as measured according to the Tensile Test Method.

In one example, the article exhibits a geometric mean tensile strength of about 100 g/in or more according to the Tensile Test Method.

In one example, the article exhibits a water content of from about 0% to about 20% and/or from about 0% to about 5% as measured according to the Water Content Test Method. In one example, the article exhibits a water content of from about 2% to about 15% and/or from about 2% to about 10% and/or from about 5% to about 10% as measured according to the Water Content Test Method.

In one example, the fibrous elements and/or fibrous structure and/or article of the present invention is “soap free”. In one example, the fibrous elements and/or fibrous structure and/or article of the present invention comprises less than about 5%, or less than about 3%, or less than about 2% of one or more lathering surfactants or soaps. In one example, the fibrous elements and/or fibrous structure and/or article is free or substantially free of lathering surfactants or soaps. A lathering surfactant is defined as a surfactant which when combined with water and mechanically agitated generate a foam or lather. Lathering surfactants include anionic and amphoteric lathering surfactants and mixtures thereof. Anionic lathering surfactants include sarcosinates, sulfates, sulfonate, isethionate, taurates, phosphates, lactylates, glutamates, alkali metal salts of fatty acids (i.e. soaps) having from 8 to 24 carbons, and mixtures thereof. “Free of soap” means that the fibrous elements and/or fibrous structure and/or article contains less than 5% and/or less than about 3%, or less than about 2% and/or less than 1% and/or about 0% or 0% by weight of salts of fatty acids.

The fibrous elements and/or fibrous structure and/or article may comprise less than 5% and/or less than 1% by weight and/or less than about 0% by weight, for example substantially free of soap (i.e. salts of fatty C4 to C30 acids) or lathering surfactant as defined hereinabove.

As indicated by the title, soaps are salts of fatty acids. Fatty acids are carboxylic acids, i.e., molecules with a C O OH acid fragment (circled in the structures below) attached to a long hydrocarbon tail (denoted below). The hydrocarbon portion could be saturated, containing only single carbon-carbon bonds as in stearic acid, or it might be unsaturated, as in oleic acid. Unsaturated fatty acids have carbon-carbon double bonds, either in one place (monounsaturated) or at multiple sites in the chain (polyunsaturated). In either case, the hydrocarbon chain is nonpolar. In one example, the fatty acid is stearic acid, a saturated fatty acid. In one example, the fatty acid is oleic acid, a monounsaturated fatty acid.

As shown in FIG. 4, an example of an article 20 of the present invention, for example a multi-ply fibrous structure according to the present invention may comprise two or more different fibrous structure plies 22, 24 (in the z-direction of the article 20 of fibrous elements 10, in this case filaments, of the present invention that form the fibrous structures of the article 20). The fibrous elements 10 in ply 22 may be the same as or different from the fibrous elements 10 in ply 24. Each ply 22, 24 may comprise a plurality of identical or substantially identical or different fibrous elements 10. For example, fibrous elements that may release their active agents at a faster rate than others within the article 20 and/or one or more fibrous structure plies 22, 24 of the article 20 may be positioned as an external surface of the article 20. The plies 22 and 24 may be associated with each other for example by mechanical entanglement at their interface between the two plies and/or by thermal or adhesive bonding and/or rodding and/or embossing and/or pressure bonding. In one example, a plurality of fibrous elements may be directly deposited, such as by spinning, directly upon an existing fibrous structure such that an additional layer of the fibrous structure is formed by the additional fibrous elements.

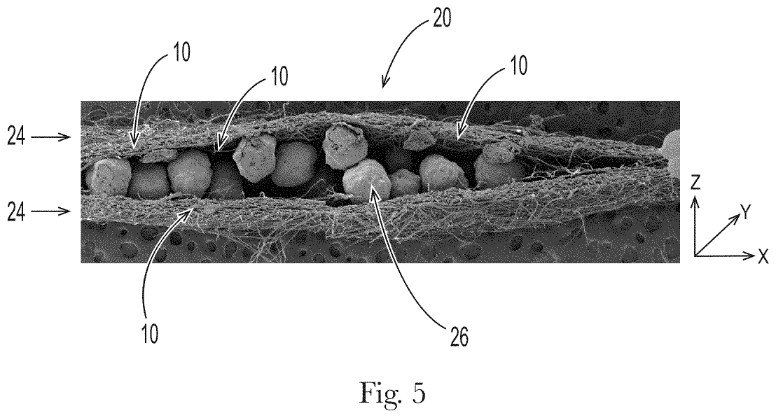

As shown in FIG. 5, another example of an article 20, for example a fibrous structure according to the present invention comprises a first fibrous structure ply 22 comprising a plurality of fibrous elements 10 of the present invention, for example filaments, a second fibrous structure ply 24 comprising a plurality of fibrous elements 10 of the present invention, for example filaments, and a plurality of particles 26 or a particle layer positioned between the first and second fibrous structure plies 22 and 24.

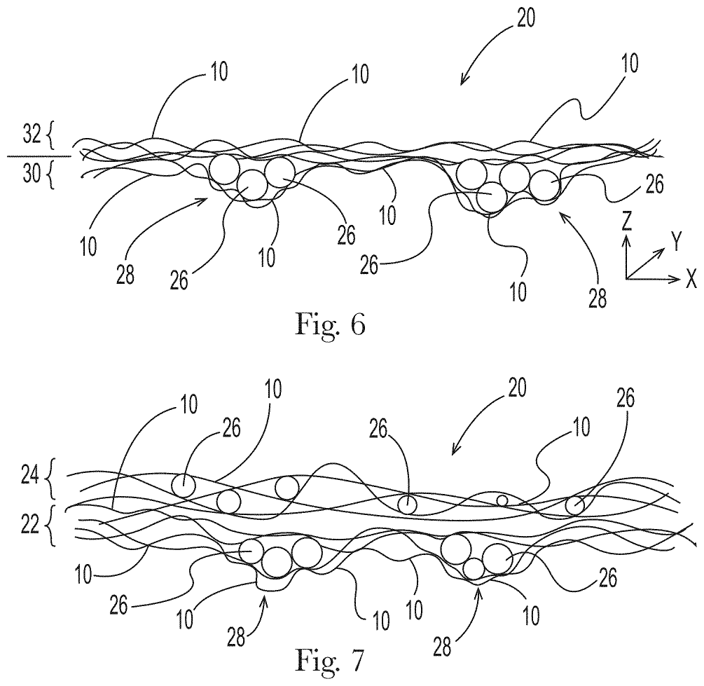

As shown in FIG. 6, another example of an article 20, for example a fibrous structure (multi-layered fibrous structure) of the present invention comprises a first fibrous structure layer 30 comprising a plurality of fibrous elements 10 of the present invention, for example filaments, wherein the first fibrous structure layer 30 comprises one or more pockets 28 (also referred to as recesses, unfilled domes, or deflected zones), which may be in an irregular pattern or a non-random, repeating pattern. One or more of the pockets 28 may contain one or more particles 26. The article 20 in this example further comprises a second fibrous structure layer 32 that is associated with the first fibrous structure layer 30 such that the particles 26 are entrapped in the pockets 28. A similar article can be formed by depositing a plurality of particles in pockets of a first ply of fibrous structure comprising a plurality of fibrous elements and then associating a second ply of fibrous structure comprising a plurality of fibrous elements such that the particles are entrapped within the pockets of the first ply. In one example, the pockets may be separated from the fibrous structure to produce discrete pockets.

As shown in FIG. 7, another example of an article 20, for example a multi-ply fibrous structure of the present invention comprises a first ply 22 of a fibrous structure according to FIG. 5 above and a second ply 24 of fibrous structure associated with the first ply 22, wherein the second ply 24 comprises a plurality of fibrous elements, for example filaments 10, and a plurality of particles 26 dispersed, in this case randomly, in the x, y, and z axes, throughout the article 20.

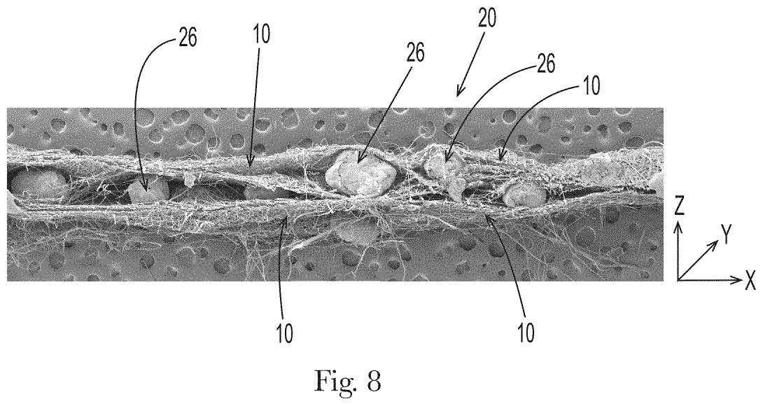

As shown in FIG. 8, another example of an article 20, for example a fibrous structure of the present invention comprises a plurality of fibrous elements 10, for example filaments, such as active agent-containing filaments, and a plurality of particles 26, for example active agent-containing particles, that have been coformed with the plurality of fibrous elements 10 such that the particles 26 are dispersed, in this case randomly, in the x, y, and z axes, throughout the fibrous structure of the article 20.

In one example, the fibrous structure may comprise discrete regions of fibrous elements that differ from other parts of the fibrous structure.

The fibrous structure of the present invention may be used as is or may be coated with one or more active agents.

### Fibrous Elements

The fibrous elements of the present invention are water-insoluble. In one example, the fibrous elements comprise one or more active agents that are releasable from the fibrous element, such as when the fibrous element and/or fibrous structure comprising the fibrous element is exposed to conditions of intended use. In addition to the one or more active agents, the fibrous elements may comprise one or more active agents. The one or more active agents may be releasable from the fibrous element when exposed to conditions of intended use.

In one example, the total level of the one or more active agents present in the fibrous elements and/or articles is 70% or greater and/or 75% or greater and/or 80% or greater and/or greater than 85% and/or greater than 90% and/or greater than 95% and/or greater than 96% and/or greater than 97% and/or greater than 98% and/or greater than 99% and/or to about 100% by weight on a dry fibrous element and/or dry fibrous structure and/or dry article basis. In one example, one or more auxiliary ingredients, for example one or more filament-forming materials, such as one or more structurants, may be present in the fibrous elements and/or articles at a total level of 30% or less and/or 25% or less and/or 20% or less and/or less than 15% and/or less than 10% and/or less than 5% and/or less than 4% and/or less than 3% and/or less than 2% and/or less than 1% and/or to about 0% by weight on a dry fibrous element and/or dry fibrous structure and/or dry article basis.

In one example, the fibrous elements exhibit a diameter of less than 100 μm and/or less than 75 μm and/or less than 50 μm and/or less than 25 μm and/or less than 10 μm and/or less than 5 μm and/or less than 1 μm as measured according to the Diameter Test Method described herein. In another example, the fibrous element of the present invention exhibits a diameter of greater than 1 μm as measured according to the Diameter Test Method described herein. The diameter of a fibrous element of the present invention may be used to control the rate of release of one or more active agents present in the fibrous element and/or the rate of loss and/or altering of the fibrous element's physical structure.

The fibrous element may comprise two or more different active agents. In one example, the fibrous element comprises two or more different active agents, wherein the two or more different active agents are compatible with one another. In another example, the fibrous element comprises two or more different active agents, wherein the two or more different active agents are incompatible with one another.

In one example, the fibrous element may comprise an active agent within the fibrous element and an active agent on an external surface of the fibrous element, such as an active agent coating on the fibrous element. The active agent on the external surface of the fibrous element may be the same or different from the active agent present in the fibrous element. If different, the active agents may be compatible or incompatible with one another.

In another example, the fibrous structure or article of the present invention may comprise a coating on the external fibrous elements or filaments on one of the surfaces of the plies of the article. The coating may be applied to a surface of a ply and the surface with the coating may be an outer surface of the overall article or may be a surface internal to the article. Placement of the coating depends upon the benefit or active agent desired to be delivered. For example, coatings on an outer surface ply of the article would be more readily visible to a consumer, as it is on a consumer viewable surface. A coating on internal surface ply of the article may be less visible, as it may be hidden from direct view by a consumer. Placement of the coating on an internal surface and/or an outer surface of the article will be achieved as part of the article making process. A coating on an internal surface ply may be different or the same as coatings on the outer surface of the article. In one example, an article may have coatings on outer surfaces and/or internal surfaces of the article. In another example, an article may have coatings on outer surfaces and/or internal surfaces of plies making up the article. In yet another example, an article may have a silicone active agent comprising a coating or an aminosilicone comprising a coating on outer surfaces and/or internal surfaces of plies making up the article.

In one example, one or more active agents may be uniformly distributed or substantially uniformly distributed throughout the fibrous element. In another example, one or more active agents may be distributed as discrete regions within the fibrous element. In still another example, at least one active agent is distributed uniformly or substantially uniformly throughout the fibrous element and at least one other active agent is distributed as one or more discrete regions within the fibrous element. In still yet another example, at least one active agent is distributed as one or more discrete regions within the fibrous element and at least one other active agent is distributed as one or more discrete regions different from the first discrete regions within the fibrous element.

### Active Agents

Non-limiting examples of suitable active agents for use in the fibrous elements and/or articles of the present invention include fatty amphiphile active agents, such as fatty alcohols, fatty acids, fatty quaternary ammonium compounds and mixtures thereof, surfactant, such as cationic surfactants, such as quaternary ammonium compounds, and shave prep active agents.

In one example, the fibrous elements and/or fibrous element-forming compositions and/or articles of the present invention comprise one or more fatty amphiphile active agents, for example one or more fatty acids and/or one or more fatty quaternary ammonium compounds and/or one or more fatty alcohols, and one or more quaternary ammonium compound active agents.

In one example, the fibrous elements and/or fibrous element-forming compositions and/or articles of the present invention comprise one or more fatty alcohols and one or more quaternary ammonium compounds in a weight ratio of greater than 1:1 and/or greater than 1.5:1 and/or greater than 1.75:1 and/or greater than 1.9:1.

In one example, the fibrous elements and/or fibrous element-forming compositions and/or articles of the present invention comprise one or more fatty acids and one or more quaternary ammonium compounds. In one example, the article of the present invention comprises one or more fatty acids and one or more quaternary ammonium compounds in a weight ratio of greater than 1:1 and/or greater than 1.5:1 and/or greater than 1.75:1 and/or greater than 1.9:1.

In one example, the water-insoluble fibrous element and/or water-insoluble article and/or fibrous element-forming composition comprises one or more active agents selected from the group consisting of: fatty acids, fatty acid derivatives, sulfonic acid derivatives, quaternary ammonium compounds, tertiary amines and salts thereof, nonionic surfactants, fatty alcohols, and mixtures thereof.

In one example, the water-insoluble fibrous element and/or water-insoluble article and/or fibrous element-forming composition comprises one or more active agents comprising a fatty acid selected from the group consisting of: myristic acid, stearic acid, isostearic acid, cetearic acid, dodecanoic acid, linoleic acid, oleic acid, palmitic acid, lauric acid, and mixtures thereof.

In one example, the water-insoluble fibrous element and/or water-insoluble article and/or fibrous element-forming composition comprises one or more active agents comprising a quaternary ammonium compound selected from the group consisting of: di(tallowyloxyethyl)hydroxyethylmethylammoniummethylsulfate, dimethyl bis(stearoyl oxyethyl)ammonium chloride, dimethyl bis(tallowyloxyethyl)ammonium chloride, dimethyl bis(tallowyloxyisopropyl) ammonium methylsulfate, behentrimonium methosulfate, behentrimonium chloride, behenamidopropyl dimethylamine and mixtures thereof.

In one example, the water-insoluble fibrous element and/or water-insoluble article and/or fibrous element-forming composition comprises one or more active agents comprises a fatty alcohol selected from the group consisting of: cetyl alcohol, stearyl alcohol, behenyl alcohol, lauryl alcohol, myristic alcohol, isostearyl alcohol, arachidyl alcohol, and mixtures thereof.

The water-insoluble fibrous elements of the present invention may comprise greater than 60% and/or greater than 70% and/or greater than 75% and/or greater than 80% and/or greater than 85% and/or greater than 90% and/or greater than 95% and/or greater than 97% to about 100% and/or to about 99% by weight of the dry fibrous elements of the one or more active agents.

Non-limiting examples of other active agents that are useful in the fibrous elements and/or fibrous element-forming compositions and/or articles of the present invention are described in U.S. Pat. No. 4,103,047, Zaki et al., issued Jul. 25, 1978; U.S. Pat. No. 4,237,155, Kardouche, issued Dec. 2, 1980; U.S. Pat. No. 3,686,025, Morton, issued Aug. 22, 1972; U.S. Pat. No. 3,849,435, Diery et al., issued Nov. 19, 1974: and U.S. Pat. No. 4,073,996, Bedenk, issued Feb. 14, 1978; said patents are hereby incorporated herein by reference.

Quaternary Ammonium Compound Active Agents

In one example, the fibrous elements and/or fibrous element-forming compositions and/or articles of the present invention comprise a quaternary ammonium compound. Non-limiting examples of quaternary ammonium compounds include alkylated quaternary ammonium compounds, ring or cyclic quaternary ammonium compounds, aromatic quaternary ammonium compounds, diquaternary ammonium compounds, alkoxylated quaternary ammonium compounds, amidoamine quaternary ammonium compounds, ester quaternary ammonium compounds, and mixtures thereof. See U.S. Patent Pub. 2005/0192207 at 57-66.

Non-limiting examples of suitable quaternary ammonium compounds include cationic active agents and their salts such as dialkyl dimethylammonium chlorides, methylsulfates and ethylsulfates wherein the alkyl groups can be the same or different and contain from about 12 to about 22 carbon atoms. Non-limiting examples of such cationic active agents include ditallowalkyldimethylammonium methylsulfate (DTDMAMS), distearyldimethylammonium methylsulfate, dipalmityldimethylammonium methylsulfate and dibehenyldimethylammonium methylsulfate. In one example, the quaternary ammonium compound may comprise distearyldimonium chloride.

Another example of a suitable active agents is an ester quaternary ammonium compound (EQA) selected from Formulas IA, IB, II, III, IV, and mixtures thereof.
Formula IA comprises:

[(R¹)₄₋ₚ—N⁺—((CH₂)ᵥ—Y—R²)ₚ]X⁻

wherein each Y═—O—(O)C—, or —C(O)—O—; p=1 to 3; each v=is an integer from 1 to 4, and mixtures thereof; each R¹ substituent is a short chain C₁-C₆, and/or C₁-C₃, alkyl group, e.g., methyl, ethyl, propyl, and the like, benzyl and mixtures thereof; each R² is a long chain, saturated and/or unsaturated (Iodine Value of from about 3 to about 60), C₈-C₃₀ hydrocarbyl, or substituted hydrocarbyl substituent and mixtures thereof; and the counterion, X⁻, can be any softener-compatible anion, for example, methylsulfate, ethylsulfate, chloride, bromide, formate, sulfate, lactate, nitrate, benzoate, and the like, such as methylsulfate.

It will be understood that substituents R¹ and R² of Formula IA can optionally be substituted with various groups such as alkoxyl or hydroxyl groups. In one example, Formula IA compounds are diester quaternary ammonium salts (DEQA). At least about 25% of the DEQA is in the diester form, and from 0% to about 40% and/or less than about 30% and/or less than about 20%, can be EQA monoester (e.g., only one —Y—R² group).

Formula IB comprises:

[(R₁)₄₋ₚ—N⁺—((CH₂CHR³)ᵥ—Y—R²)ₚ]X⁻

wherein each Y═—O—(O)C—, or —C(O)—O—; p=1 to 3; each v=is an integer from 1 to 4, and mixtures thereof; each R¹ substituent is a short chain C₁-C₆, and/or C₁-C₃, alkyl group, e.g., methyl, ethyl, propyl, and the like, benzyl and mixtures thereof; each R² is a long chain, saturated and/or unsaturated (Iodine Value of from about 3 to about 60), C₈-C₃₀ hydrocarbyl, or substituted hydrocarbyl substituent and mixtures thereof; each R³ substituent is a short chain C₁-C₆ including benzyl, and/or C₁-C₃ alkyl group e.g., methyl, ethyl, propyl, and/or C₁-C₂ e.g., methyl, ethyl, and mixtures thereof; and the counterion, X⁻, can be any softener-compatible anion, for example, methylsulfate, ethylsulfate, chloride, bromide, formate, sulfate, lactate, nitrate, benzoate, and the like, such as methylsulfate.

It will be understood that substituents R¹ and R² of Formula IB can optionally be substituted with various groups such as alkoxyl or hydroxyl groups. In one example, Formula IB compounds are diester quaternary ammonium salts (DEQA). At least about 25% of the DEQA is in the diester form, and from 0% to about 40% and/or less than about 30% and/or less than about 20%, can be EQA monoester (e.g., only one —Y—R² group).

As used herein, when the diester is specified, it will include the monoester that is normally present. The level of monoester present can be controlled in the manufacturing of the EQA.

The following are non-limiting examples of EQA Formula IA or IB (wherein all long-chain alkyl substituents are straight-chain):

Saturated

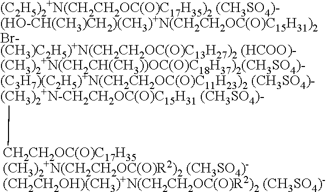

where —C(O)R² is derived from saturated tallow.

Unsaturated

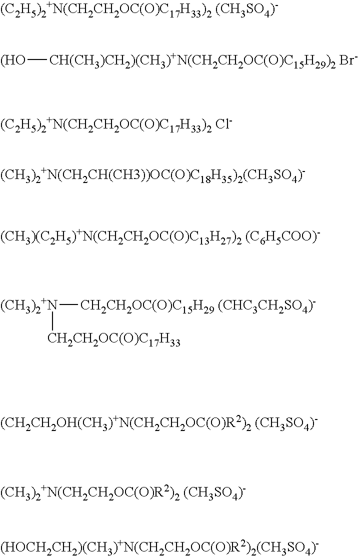

where —C(O)R² is derived from partially hydrogenated tallow or modified tallow having the characteristics set forth herein.

In addition to Formula IA and IB compounds, the compositions and articles of the present invention comprise EQA compounds of Formula II:

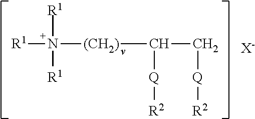

wherein, for any molecule: each Q is —O—C(O)— or —C(O)—O—; each R¹ is C₁-C₄ alkyl or hydroxy alkyl; R² and v are defined hereinbefore for Formula IA and IB; for example wherein R¹ is a methyl group, v is 1, Q is —O—C(O)—, each R² is C₁₄-C₁₈, and X⁻ is methyl sulfate.

The straight or branched alkyl or alkenyl chains, R², have from about 8 to about 30 carbon atoms and/or from about 14 to about 18 carbon atoms and/or straight chains having from about 14 to about 18 carbon atoms.

Tallow is a convenient and inexpensive source of long chain alkyl and alkenyl materials.

A specific example of a Formula II EQA compound suitable for use in the present invention is: 1,2-bis(tallowyl oxy)-3-trimethyl ammoniopropane methylsulfate (DTTMAPMS).

Other examples of suitable Formula II EQA compounds of this invention are obtained by, e.g., replacing “tallowyl” in the above compounds with, for example, cocoyl, lauryl, oleyl, stearyl, palmityl, or the like; replacing “methyl” in the above compounds with ethyl, propyl, isopropyl, butyl, isobutyl, t-butyl, or the hydroxy substituted analogs of these radicals; and/or replacing “methylsulfate” in the above compounds with chloride, ethylsulfate, bromide, formate, sulfate, lactate, nitrate, and the like, for example methylsulfate.

In addition to Formula IA and IB and Formula II compounds, the articles of the present invention may comprise EQA compounds of Formula III:

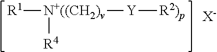

wherein R⁴=a short chain C₁-C₄ alcohol; p is 2; R¹, R², v, Y, and X are as previously defined for Formula IA and IB.

A specific example of a Formula III compound suitable for use as an active agent is N-methyl-N,N-di-(2-(C₁₄-C₁₈-acyloxy) ethyl), N-2-hydroxyethyl ammonium methylsulfate. An example of such as compound is N-methyl, N,N-di-(2-oleyloxyethyl)N-2-hydroxyethyl ammonium methylsulfate.

Active agents of the present invention may also comprise Formula IV compounds:

[(R¹)₄₋ₚ—N⁺—((CH₂)ᵥ—Y″—R²)ₚ]X⁻

wherein R¹, R², p, v, and X⁻ are previously defined in Formula IA and IB; and

[(R¹)₄₋ₚ—N⁺—((CH₂)ᵥ—Y—R²)ₚ]X⁻

and mixtures thereof, wherein at least one Y″ group is

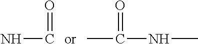

An example of this compound is methyl bis (oleyl amidoethyl) 2-hydroxyethyl ammonium methyl sulfate.

In one example, the active agent of the present invention is a quaternary ammonium compound.

The quaternary ammonium compounds herein can be prepared by standard esterification and quaternization reactions, using readily available starting materials. General methods for preparation are disclosed in U.S. Pat. No. 4,137,180, which is incorporated herein by reference.

Tertiary Amines and Salts Thereof

Another active agent useful in the fibrous elements and/or articles of the present invention is a carboxylic acid salt of a tertiary amine and/or ester amine having the formula:

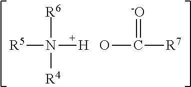

wherein R⁵ is a long chain aliphatic group containing from about 8 to about 30 carbon atoms; R⁶ and R⁴ are the same or different from each other and are selected from the group consisting of aliphatic groups containing from about 1 to about 30 carbon atoms, hydroxyalkyl groups of the Formula R⁸OH wherein R⁸ is an alkylene group of from about 2 to about 30 carbon atoms, and alkyl ether groups of the formula R⁹O(CₙH₂ₙO)ₘ wherein R⁹ is alkyl and alkenyl of from about 1 to about 30 carbon atoms and hydrogen, n is 2 or 3, and m is from about 1 to about 30; wherein R⁴, R⁵, R⁶, R⁸, and R⁹ chains can be ester interrupted groups; and wherein R⁷ is selected from the group consisting of unsubstituted alkyl, alkenyl, aryl, alkaryl and aralkyl of about 8 to about 30 carbon atoms, and substituted alkyl, alkenyl, aryl, alkaryl, and aralkyl of from about 1 to about 30 carbon atoms wherein the substituents are selected from the group consisting of halogen, carboxyl, and hydroxyl, said composition having a thermal softening point of from about 35° C. to about 100° C. In one example, any of R⁴, R⁵, R⁶, R⁷, R⁸, and/or R⁹ chains can contain unsaturation.

In one example, R⁵ is an aliphatic chain containing from about 12 to about 30 carbon atoms, R⁶ is an aliphatic chain of from about 1 to about 30 carbon atoms, and R⁴ is an aliphatic chain of from about 1 to about 30 carbon atoms. In one example, suitable tertiary amines for static control performance are those containing unsaturation; e.g., oleyldimethylamine and/or soft tallowdimethylamine.

Examples of suitable tertiary amines as starting material for the reaction between the amine and carboxylic acid to form the tertiary amine salts are: lauryldimethylamine, myristyldimethyl-amine, stearyldimethylamine, tallowdimethylamine, coconutdimethylamine, dilaurylmethylamine, distearylmethylamine, ditallowmethylamine, oleyldimethylamine, dioleylmethylamine, lauryldi(3-hydroxypropyl)amine, stearyldi(2-hydroxyethyl)amine, trilaurylamine, laurylethylmethylamine, and

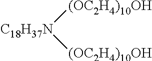

Non-limiting examples of suitable fatty acids are those wherein R⁷ is a long chain, unsubstituted alkyl or alkenyl group of from about 8 to about 30 carbon atoms and/or from about 11 to about 17 carbon atoms.

Examples of specific carboxylic acids as a starting material are: formic acid, acetic acid, lauric acid, myristic acid, palmitic acid, stearic acid, oleic acid, oxalic acid, adipic acid, 12-hydroxy stearic acid, benzoic acid, 4-hydroxy benzoic acid, 3-chloro benzoic acid, 4-nitro benzoic acid, 4-ethyl benzoic acid, 4-(2-chloroethyl)benzoic acid, phenylacetic acid, (4-chlorophenyl)acetic acid, (4-hydroxyphenyl)acetic acid, and phthalic acid.

Non-limiting examples of suitable carboxylic acids are stearic, oleic, lauric, myristic, palmitic, and mixtures thereof.

The amine salt can be formed by a simple addition reaction, well known in the art and disclosed in U.S. Pat. No. 4,237,155, Kardouche, issued Dec. 2, 1980, which is incorporated herein by reference. Excessive levels of free amines may result in odor problems, and generally free amines provide poorer softening performance than the amine salts.

Non-limiting examples of amine salts for use herein are those wherein the amine moiety is a C₈-C₃₀ alkyl or alkenyl dimethyl amine and/or a di-C₈-C₃₀ alkyl or alkenyl methyl amine, and the acid moiety is a C₈-C₃₀ alkyl and/or alkenyl monocarboxylic acid. The amine and the acid, respectively, used to form the amine salt will often be of mixed chain lengths rather than single chain lengths, since these materials are normally derived from natural fats and oils, or synthetic processed which produce a mixture of chain lengths. Also, it is often desirable to utilize mixtures of different chain lengths in order to modify the physical or performance characteristics of the softening composition.

Specific examples of amine salts for use in the present invention are oleyldimethylamine stearate, stearyldimethylamine stearate, stearyldimethylamine myristate, stearyldimethylamine oleate, stearyldimethylamine palmitate, distearylmethylamine palmitate, distearylmethylamine laurate, and mixtures thereof. In one example, a mixture of amine salts is oleyldimethylamine stearate and distearylmethylamine myristate, in a ratio of 1:10 to 10:1 and/or about 1:1.

Sulfonic Acid Fatty Amine Salts

Other fatty amine salts can be used in the present invention. These salts are similar to those previously described but replacing the carboxylic acid with a sulfonic acid derivative. The amine salt can be formed by a simple addition reaction, well known in the art and disclosed in U.S. Pat. No. 4,861,502, Caswell issued Aug. 29, 1989, which is incorporated herein by reference. Such sulfonic acid derivates include but not limited to methylsulfonic acid, benzenesulfonic acid, toluensulfonic acid, cumenesulfonic and mixtures thereof.

Nonionic Active Agents

Non-limiting examples of suitable nonionic active agents for use in the fibrous elements and/or articles of the present invention have an HLB of from about 2 to about 9, and more typically from about 3 to about 7. In general, the materials selected should be relatively crystalline and higher melting, (e.g., >25° C.).

The level of optional nonionic active agents in the article is typically from about 0.1% to about 50% and/or from about 5% to about 30%.

Non-limiting examples of suitable nonionic active agents are fatty acid partial esters of polyhydric alcohols, or anhydrides thereof, wherein the alcohol or anhydride contains from about 2 to about 18 and/or from about 2 to about 8 carbon atoms, and each fatty acid moiety contains from about 8 to about 30 and/or from about 12 to about 20 carbon atoms. Typically, such nonionic active agents contain from about one to about 3 and/or about 2 fatty acid groups per molecule.

The polyhydric alcohol portion of the ester can be ethylene glycol, glycerol, poly (e.g., di-, tri-, tetra, penta-, and/or hexa-) glycerol, xylitol, sucrose, erythritol, pentaerythritol, sorbitol or sorbitan.

The fatty acid portion of the ester is normally derived from fatty acids having from about 8 to about 30 and/or from about 12 to about 22 carbon atoms. Typical examples of said fatty acids being lauric acid, myristic acid, palmitic acid, stearic acid, oleic acid, and behenic acid.

Non-limiting examples of suitable nonionic active agents for use in the present invention are C₁₀-C₂₆ acyl sorbitan esters and polyglycerol monostearate. Sorbitan esters are esterified dehydration products of sorbitol. The sorbitan ester may comprise a member selected from the group consisting of C₁₀-C₂₆ acyl sorbitan monoesters and/or C₁₀-C₂₆ acyl sorbitan diesters and/or ethoxylates of said esters wherein one or more of the unesterified hydroxyl groups in said esters contains from about 1 to about 6 oxyethylene units, and mixtures thereof. For the purpose of the present invention, sorbitan esters containing unsaturation (e.g., sorbitan monooleate) can be utilized.

Sorbitol, which is typically prepared by the catalytic hydrogenation of glucose, can be dehydrated in well-known fashion to form mixtures of 1,4- and 1,5-sorbitol anhydrides and small amounts of isosorbides. (See U.S. Pat. No. 2,322,821, Brown, issued Jun. 29, 1943, incorporated herein by reference.)

The foregoing types of complex mixtures of anhydrides of sorbitol are collectively referred to herein as “sorbitan.” It will be recognized that this “sorbitan” mixture will also contain some free, uncyclized sorbitol.

In one example, the sorbitan active agents of the type employed herein can be prepared by esterifying the “sorbitan” mixture with a fatty acyl group in standard fashion, e.g., by reaction with a fatty acid halide, fatty acid ester, and/or fatty acid. The esterification reaction can occur at any of the available hydroxyl groups, and various mono-, di-, etc., esters can be prepared. In fact, mixtures of mono-, di-, tri-, etc., esters almost always result from such reactions, and the stoichiometric ratios of the reactants can be simply adjusted to favor the desired reaction product.

For commercial production of the sorbitan ester materials, etherification and esterification are generally accomplished in the same processing step by reacting sorbitol directly with fatty acids. Such a method of sorbitan ester preparation is described more fully in MacDonald, “Emulsifiers: Processing and Quality Control”, Journal of the American Oil Chemists' Society, Vol. 45, October 1968. Details, including formula, of the examples of sorbitan esters can be found in U.S. Pat. No. 4,128,484, incorporated hereinbefore by reference.

Certain derivatives of the sorbitan esters herein, especially the “lower” ethoxylates thereof (i.e., mono-, di-, and tri-esters wherein one or more of the unesterified —OH groups contain one to about twenty oxyethylene moieties (Tweens®) are also useful in the articles of the present invention. Therefore, the term “sorbitan ester” is intended to include such derivatives.

For the purposes of the present invention, in one example, a significant amount of di- and tri-sorbitan esters are present in the ester mixture. In another example, an ester mixture may have from about 20-50% mono-ester, about 25-50% di-ester and about 10-35% of tri- and tetra-esters. Material which is sold commercially as sorbitan mono-ester (e.g., monostearate) typically contains significant amounts of di- and tri-esters. A typical analysis of commercial sorbitan monostearate indicates that it comprises about 27% mono-, about 32% di- and about 30% tri- and tetra-esters. Mixtures of sorbitan stearate and sorbitan palmitate having stearate/palmitate weight ratios varying between 10:1 and 1:10, and 1,5-sorbitan esters are also useful. In addition, both the 1,4- and 1,5-sorbitan esters are useful herein.

Other useful alkyl sorbitan esters for use as active agents herein include sorbitan monolaurate, sorbitan monomyristate, sorbitan monopalmitate, sorbitan monobehenate, sorbitan monooleate, sorbitan dilaurate, sorbitan dimyristate, sorbitan dipalmitate, sorbitan distearate, sorbitan dibehenate, sorbitan dioleate, and mixtures thereof, and mixed tallowalkyl sorbitan mono- and di-esters. Such mixtures are readily prepared by reacting the foregoing hydroxy-substituted sorbitans, particularly the 1,4- and 1,5-sorbitans, with the corresponding acid, ester, or acid chloride in a simple esterification reaction. It is to be recognized, of course, that commercial materials prepared in this manner will comprise mixtures usually containing minor proportions of uncyclized sorbitol, fatty acids, polymers, isosorbide structures, and the like. In the present invention, it is desirable to keep such impurities present at as low a level as practical.

The sorbitan esters employed herein may contain up to about 15% by weight of esters of the C₂₀-C₂₆, and higher, fatty acids, as well as minor amounts of C₈, and lower, fatty esters.

Glycerol and polyglycerol esters, especially glycerol, diglycerol, triglycerol, and polyglycerol mono- and/or di-esters, in one example mono- (e.g., polyglycerol monostearate with a trade name of Radiasurf 7248). Glycerol esters can be prepared from naturally occurring triglycerides by normal extraction, purification and/or interesterification processes or by esteri-fication processes of the type set forth hereinbefore for sorbitan esters. Partial esters of glycerin can also be ethoxylated to form usable derivatives that are included within the term “glycerol esters.”

Useful glycerol and polyglycerol esters include mono-esters with stearic, oleic, palmitic, lauric, isostearic, myristic, and/or behenic acids and the diesters of stearic, oleic, palmitic, lauric, isostearic, behenic, and/or myristic acids. It is understood that the typical mono-ester contains some di- and tri-ester, etc.

The “glycerol esters” also include the polyglycerol, e.g., diglycerol through octaglycerol esters. The polyglycerol polyols are formed by condensing glycerin or epichlorohydrin together to link the glycerol moieties via ether linkages. The mono- and/or diesters of the polyglycerol polyols may be used, the fatty acyl groups typically being those described hereinbefore for the sorbitan and glycerol esters.

Fatty Active Agents

The fibrous elements and/or articles of the present invention may comprise one or more fatty active agents, for example one or more high melting point fatty compounds. The high melting point fatty compound can be included in the fibrous element, article and/or fibrous element-forming composition at a level of from about 10 wt % to about 85 wt % and/or from 20 wt % to 70 wt % and/or from about 50 wt % to about 70 wt % and/or from about 10 wt % to about 20 wt % of the fibrous element and/or article and/or fibrous element-forming composition. In one example, the fatty active agent is selected from the group consisting of: fatty amphiphiles, fatty alcohols, fatty acids, fatty amides, fatty esters and mixtures thereof.

In one example, the fatty active agents have a melting point of 25° C. or higher and/or 40° C. or higher and/or 45° C. or higher and/or 50° C. or higher and/or to about 90° C. and/or to about 80° C. and/or to about 70° C. and/or to about 65° C. and are considered as high melting point fatty active agents. The fatty active agent may be used as a single compound or as a blend or mixture of at least two fatty active agents. When used as such blend or mixture, the above melting point means the melting point of the blend or mixture.

The fatty active agents useful herein may be selected from the group consisting of fatty alcohols, fatty acids, fatty alcohol derivatives, fatty acid derivatives, and mixtures thereof. It is understood by the artisan that the fatty active agents disclosed herein may in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain fatty active agents having certain required carbon atoms may have a melting point of less than the above. Such fatty active agents of low melting point (a melting point less than 25° C. and/or less than 20° C.) are not intended to be included in this section. Non-limiting examples of the high melting point fatty active agents are found in International Cosmetic Ingredient Dictionary, Fifth Edition, 1993, and CTFA Cosmetic Ingredient Handbook, Second Edition, 1992.

a. Fatty Acids

The fatty active agents may include fatty acid active agents. Typically, the fatty acid is present to improve the processability of the composition, and is admixed with any material, or materials, that are difficult to process, especially as a result of having a high viscosity. The fatty acid may provide improved viscosity and/or processability.

Non-limiting examples of suitable fatty acids are those containing a long chain, unsubstituted alkenyl group of from about 8 to about 30 carbon atoms and/or from about 11 to about 18 carbon atoms. Examples of specific carboxylic acids are: oleic acid, linoleic acid, and mixtures thereof. Although unsaturated fatty acids are desirable, the unsaturated fatty acids can also be used in combination with saturated fatty acids like stearic, palmitic, and/or lauric acids. Non-limiting examples of suitable carboxylic acids are oleic, linoleic, tallow fatty acids, and mixtures thereof.

In one example, the fatty acid is added to the quaternization reaction mixture used to form the biodegradable quaternary ammonium compounds of Formulas II, III, and/or IV as described hereinbefore to lower the viscosity of the reaction mixture to less than about 1500 cps and/or less than about 1000 cps and/or less than about 800 cps. The solvent level of added fatty acid may be from about 5% to about 30% and/or from about 10% to about 25% and/or from about 10% to about 20%. The unsaturated fatty acid can be added before the start of the quaternization reaction and/or may be added during the quaternization reaction when it is needed to reduce the viscosity which increases with increased level of quaternization. In one example, the addition occurs when at least about 60% of the product is quaternized. This allows for a low viscosity for processing while minimizing side reactions that can occur when the quaternizing agent reacts with the fatty acid. The quaternization reactions are well known and include, e.g., with respect to Formula IA and/or IB compounds, those processes described in U.S. Pat. No. 3,915,867, Kang et al., issued Oct. 28, 1975; U.S. Pat. No. 4,830,771, Ruback et al., issued May 16, 1989; and U.S. Pat. No. 5,296,622, Uphues et al., issued Mar. 22, 1994, all of said patents being incorporated herein by reference. The resulting quaternized biodegradable active agents can be used without removal of the unsaturated fatty acid, and, in fact, are more useful since the mixture is more fluid and more easily handled.

Another example of types of active agents suitable for use herein are described in detail in U.S. Pat. No. 4,661,269, Toan Trinh, Errol H. Wahl, Donald M. Swartley and Ronald L. Hemingway, issued Apr. 28, 1987, said patent being incorporated herein by reference.

b. Fatty Alcohols

Non-limiting examples of suitable fatty alcohols useful as fatty active agents are those fatty alcohols having from about 14 to about 30 carbon atoms and/or from about 16 to about 22 carbon atoms. These fatty alcohols are saturated and can be straight or branched chain alcohols.

Suitable fatty alcohols include, but are not limited to, cetyl alcohol (having a melting point of about 56° C.), stearyl alcohol (having a melting point of about 58-59° C.), behenyl alcohol (having a melting point of about 71° C.), and mixtures thereof. These fatty alcohols are known to have the above referenced melting points, however, they often have lower melting points when supplied, since such supplied products are often mixtures of fatty alcohols having alkyl chain length distributions in which the main alkyl chain is cetyl, stearyl or behenyl group. Generally, in the mixture, the weight ratio of cetyl alcohol to stearyl alcohol can be from about 1:9 to 9:1 and/or from about 1:4 to about 4:1 and/or from about 1:2.3 to about 1.5:1.

Surfactant Active Agents

The active agents of the present invention may further comprise one or more surfactants, for example one or more nonionic surfactants and/or one or more cationic surfactants. The surfactants may provide function both as an active agent as well as a fibrous element hydration controlling system.

a. Nonionic Surfactants

The total level of nonionic surfactants included in the fibrous elements and/or article may be from about 1 wt % to about 30 wt % of the composition, alternatively from about 5 wt % to about 15 wt %, and alternatively from about 5 wt % to about 10 wt %. Nonlimiting examples of noninoic surfactants include alkyl glucamides, for example lauroyl/myristoyl methyl glucamide. The alkyl glucamides contain a hydrophobic tail of about 8-18 carbons and a nonionic head group of glucamide. For the alkyl glucamides, the presence of the amide and hydroxyl groups may provide sufficient polarity that balances the hydrophobic carbon tail in such a way to permit the surfactant's solubility in the fibrous element-forming composition and/or imparts a rapid dispersion of the article's components upon exposure to water. Other suitable nonionic surfactants include, but are not limited to, reverse alkyl glucamides, betaines, such as cocamidopropyl betaines, alkyl glucosides, tertiary amino compounds, such as triethanolamine (TEA), alkanolamides, such as cocamide DEA, cocamide MEA, cocamide MIPA, isostearamide DEA, isostearamide MEA, isostearamide MIPA, lanolinamide DEA, lauramide DEA, lauramide MEA, lauramide MIPA, linoleamide DEA, linoleamide MEA, linoleamide MIPA, myristamide DEA, myristamide MEA, myristamide MIPA, Oleamide DEA, Oleamide MEA, Oleamide MIPA, palmamide DEA, palmamide MEA, palmamide MIPA, palmitamide DEA, palmitamide MEA, palm kernelamide DEA, palm kernelamide MEA, palm kernelamide MIPA, peanutamide MEA, peanutamide MIPA, soyamide DEA, stearamide DEA, stearamide MEA, stearamide MIPA, tallamide DEA, tallowamide DEA, tallowamide MEA, undecylenamide DEA, undecylenamide MEA and mixtures thereof.

b. Cationic Surfactants

When present, the cationic surfactant may be present at a level of from about 1 wt % to about 60 wt %, alternatively from about 10 wt % to about 50 wt %, alternatively from about 20 wt % to about 40 wt % of the fibrous elements and/or fibrous element-forming composition and/or article.

Cationic surfactants useful herein can be one cationic surfactant or a mixture of two or more cationic surfactants. The cationic surfactant can be selected from the group consisting of, but not limited to: a mono-long alkyl quaternized ammonium salt; a combination of a mono-long alkyl quaternized ammonium salt and a di-long alkyl quaternized ammonium salt; a mono-long alkyl amine; a combination of a mono-long alkyl amine and a di-long alkyl quaternized ammonium salt; and a combination of a mono-long alkyl amine and a mono-long alkyl quaternized ammonium salt, a tertiary amine and combinations thereof.

In one example, the water-insoluble fibrous elements and/or water-insoluble articles of the present invention comprise one or more quaternary ammonium compounds, which may be cationic surfactants, that comprise from about 8 to 30 and/or from about 8 to 24 and/or from about 8 to 22 and/or from about 8 to 20 and/or from about 10 to 18 and/or from about 12 to 18 and/or from about 14 to 18 carbon atoms.

i. Mono-Long Alkyl Amines

Non-limiting examples of mono-long alkyl amines useful herein are those having one long alkyl chain of from about 8 to 30 and/or from about 8 to 22 and/or from about 8 to 20 and/or from about 10 to 20 and/or from about 10 to 18 and/or from about 12 to 18 and/or from about 14 to 18 and/or from about 16 to 18 carbon atoms, alternatively from 16 to 24 carbon atoms, alternatively from 18 to 22 alkyl group. Mono-long alkyl amines useful herein also include mono-long alkyl amidoamines. Primary, secondary, and tertiary fatty amines are useful.

Suitable for use in the fibrous elements and/or articles of the present invention are tertiary amido amines having an alkyl group of from about 12 to about 22 carbons. Exemplary tertiary amido amines include: stearamidopropyl dimethylamine, stearamidopropyl diethylamine, stearamidoethyl diethylamine, stearamidoethyl dimethylamine, palmitamidopropyl dimethylamine, palmitamidopropyl diethylamine, palmitamidoethyl diethylamine, palmitamidoethyl dimethylamine, behenamidopropyl dimethylamine, behenamidopropyl diethylamine, behenamidoethyl diethylamine, behenamidoethyl dimethylamine, arachidamidopropyl dimethylamine, arachidamidopropyl diethylamine, arachidamidoethyl diethylamine, arachidamidoethyl dimethylamine, diethylaminoethylstearamide. Useful amines in the present invention are disclosed in U.S. Pat. No. 4,275,055, Nachtigal, et al.

These amines can be used in combination with acids such as t-glutamic acid, lactic acid, hydrochloric acid, malic acid, succinic acid, acetic acid, fumaric acid, tartaric acid, citric acid, t-glutamic hydrochloride, maleic acid, and mixtures thereof; alternatively t-glutamic acid, lactic acid, citric acid, at a molar ratio of the amine to the acid of from about 1:0.3 to about 1:2, alternatively from about 1:0.4 to about 1:1.

ii. Mono-long Alkyl Quaternized Ammonium Salts

Non-limiting examples of mono-long alkyl quaternized ammonium salts useful herein are those having one long alkyl chain which has from 12 to 30 carbon atoms, alternatively from 16 to 24 carbon atoms, alternatively a C18-22 alkyl group. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Mono-long alkyl quaternized ammonium salts useful herein are those having the following formula (V):

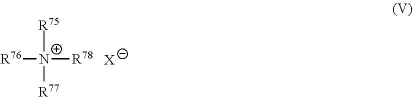

wherein one of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. One of R⁷⁵, R⁷⁶, R⁷⁷ and R⁷⁸ can be selected from an alkyl group of from 12 to 30 carbon atoms, alternatively from 16 to 24 carbon atoms, alternatively from 18 to 22 carbon atoms, alternatively 22 carbon atoms; the remainder of R⁷⁵, R⁷⁶, R⁷⁷ and R⁷⁸ can be independently selected from CH₃, C₂H₅, C₂H₄OH, and mixtures thereof; and X can be selected from the group consisting of Cl, Br, CH₃OSO₃, C₂H₅OSO₃, and mixtures thereof.

Nonlimiting examples of such mono-long alkyl quaternized ammonium salt cationic surfactants include: behenyl trimethyl ammonium salt; stearyl trimethyl ammonium salt; cetyl trimethyl ammonium salt; and hydrogenated tallow alkyl trimethyl ammonium salt.

iii. Di-long Alkyl Quaternized Ammonium Salts

When used, di-long alkyl quaternized ammonium salts can be combined with a mono-long alkyl quaternized ammonium salt and/or mono-long alkyl amine salt, at the weight ratio of from 1:1 to 1:5, alternatively from 1:1.2 to 1:5, alternatively from 1:1.5 to 1:4, in view of stability in rheology and conditioning benefits.

Non-limiting example of di-long alkyl quaternized ammonium salts useful herein are those having two long alkyl chains of from 12 to 30 carbon atoms, alternatively from 16 to 24 carbon atoms, alternatively from 18 to 22 carbon atoms. Such di-long alkyl quaternized ammonium salts useful herein are those having the formula (VI):

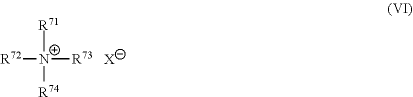

wherein two of R⁷¹, R⁷², R⁷³ and R⁷⁴ are selected from an aliphatic group of from 12 to 30 carbon atoms, alternatively from 16 to 24 carbon atoms, alternatively from 18 to 22 carbon atoms or an aromatic, alkoxy, polyoxyalkylene, alkylamido, hydroxyalkyl, aryl or alkylaryl group having up to about 30 carbon atoms; the remainder of R⁷¹, R⁷², R⁷³ and R⁷⁴ are independently selected from an aliphatic group of from 1 to about 8 carbon atoms, alternatively from 1 to 3 carbon atoms or an aromatic, alkoxy, polyoxyalkylene, alkylamido, hydroxyalkyl, aryl or alkylaryl group having up to about 8 carbon atoms; and X⁻ is a salt-forming anion selected from the group consisting of halides such as chloride and bromide, C₁-C₄ alkyl sulfate such as methosulfate and ethosulfate, and mixtures thereof. The aliphatic groups can contain, in addition to carbon and hydrogen atoms, ether linkages, and other groups such as amino groups. The longer chain aliphatic groups, e.g., those of about 16 carbons, or higher, can be saturated or unsaturated. Two of R⁷¹, R⁷², R⁷³ and R⁷⁴ can be selected from an alkyl group of from 12 to 30 carbon atoms, alternatively from 16 to 24 carbon atoms, alternatively from 18 to 22 carbon atoms; and the remainder of R⁷¹, R⁷², R⁷³ and R⁷⁴ are independently selected from CH₃, C₂H₅, C₂H₄OH, CH₂C₆H₅, and mixtures thereof.

Suitable di-long alkyl cationic surfactants include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride.

### Auxiliary Ingredients

In addition to the one or more active agents, the fibrous elements and/or fibrous element-forming compositions and/or articles of the present invention may further comprise one or more auxiliary ingredients, for example one or more structurants, such as one or more polymeric structurants. In one example, the auxiliary ingredient is derived from a non-naturally occurring polymer, in other words, the auxiliary ingredient isn't derived from starch or cellulose for example.

Non-limiting examples of suitable auxiliary ingredients, for example structurants, are selected from the group consisting of: polymeric structurants, inorganic structurants, and mixtures thereof. In one example, the auxiliary ingredient, for example structurant, comprises a polymeric structurant selected from the group consisting of: polyvinylpyrrolidone, copolymers of vinylpyrrolidone, polydimethylacrylamide, copolymers of dimethylacrylamide, and mixtures thereof. In one example, the structurant comprises polyvinylpyrrolidone. In one example, the structurant comprises polydimethylacrylamide. In one example, the structurant comprises an inorganic structurant selected from the group consisting of clays, silica, and mixtures thereof.

The one or more auxiliary ingredients, for example one or more structurants, when present, may be dispersed in, for example homogeneously, the one or more active agents within the filament-forming composition and/or fibrous element and/or fibrous structure and/or article.

When present, the one or more auxiliary ingredients may be present in the filament-forming composition and/or fibrous element and/or fibrous structure and/or article at a total level of less than 20% or less and/or less than 15% and/or less than 10% and/or less than 5% and/or less than 4% and/or less than 3% and/or less than 2% and/or less than 1% and/or about 0% by weight on a dry filament-forming composition and/or a dry fibrous element and/or dry fibrous structure and/or dry article basis.

Non-limiting examples of suitable structurants include polymeric structurants selected from the group consisting of: polyacrylic acid and its copolymers, polyvinylpyrrolidone (PVP) and its copolymers, for example cationic copolymers thereof, such as polyvinylpyrrolidone/trimethyl amino ethyl methacrylate (PVP/TMAEMA), polyvinylpyrrolidone/dimethyl aminoethyl methacrylate (PVP/DMAEMA) and polyvinylpyrrolidone/dimethyl aminopropylmethyacrylate (PVP/DMAPMA), polyacrylamide and its copolymers, polyoxazoline and its copolymers, such as poly(2-ethyl oxazoline), polyvinylmethyl ether, polyethyleneimine, polymethacrylic acid, other water soluble acrylic polymers such as N-isopropyl acrylamide, N—N-dimethylacrylamide, polyvinyloxazolidone, polycaprolactam, polystyrene sulfonate, polyvinyl formamide, polyvinyl amine, alkylated vinyl pyrrolidone, vinyl caprolactam, vinyl valerolactam, vinyl imidazole, acrylic acid, methacrylate, acrylamide, methacrylamide, dimethacrylamide, alkylaminomethacrylate, and alkylaminomethacrylamide monomers, and combinations thereof. For purposes of clarity, the use of the term “copolymer” is intended to convey that the vinyl pyrrolidone monomer can be copolymerized with other non-limiting monomers such as vinyl acetate, alkylated vinyl pyrrolidone, vinyl caprolactam, vinyl valerolactam, vinyl imidazole, acrylic acid, methacrylate, acrylamide, methacrylamide, dimethacrylamide, alkylaminomethacrylate, and alkylaminomethacrylamide monomers.

In one example, the structurant comprises a polymeric structurant selected from the group consisting of: polyvinylpyrrolidone, copolymers of vinylpyrrolidone, polydimethylacrylamide, copolymers of dimethylacrylamide, and mixtures thereof. In one example, the structurant comprises polyvinylpyrrolidone. In another example, the structurant comprises polydimethylacrylamide.

In one example, at least one of the one or more auxiliary ingredients, for example a structurant, such as a polymeric structurant, exhibits a weight average molecular weight of from about 10,000 to about 3,000,000 g/mol and/or from about 20,000 to about 2,500,000 g/mol and/or from about 30,000 to less than 2,000,000 g/mol and/or from about 40,000 to about 1,700,000 g/mol.

### Fibrous Element Hydration Controlling System

In addition to the active agents and auxiliary ingredients, the fibrous elements and/or fibrous element-forming compositions and/or articles of the present invention comprise a fibrous element hydration controlling system. Such as fibrous element hydration controlling system may be present in the fibrous elements and/or fibrous element-forming composition and ultimately in articles produced therefrom and/or may be an external fibrous element hydration controlling system that is no present in the fibrous elements nor the fibrous element-forming compositions.

Non-limiting examples of suitable fibrous element hydration controlling systems for use in the fibrous elements and/or fibrous element-forming compositions and ultimately in the articles produced therefrom include quaternary ammonium compounds, for example C₈-C₂₂ quaternary ammonium compounds, which may also function as cationic surfactants, surfactants, for example nonionic surfactants, salts and salt-generating systems, for example effervescent systems, and mixtures thereof.

a. Quaternary Ammonium Compounds

In one example, the fibrous element hydration controlling system comprises one or more quaternary ammonium compounds. In one example, the fibrous element hydration controlling system comprises one or more C₈-C₂₂ quaternary ammonium compounds and/or one or more C₈-C₂₀ quaternary ammonium compounds and/or one or more C₁₀-C₁₈ quaternary ammonium compounds and/or one or more C₁₂-C₁₈ quaternary ammonium compounds and/or one or more C₁₄-C₁₈ quaternary ammonium compounds and/or one or more C₁₆-C₁₈ quaternary ammonium compounds.

In one example, the fibrous element hydration controlling system comprises a quaternary ammonium compound selected from the group consisting of: behentrimonium methosulfate, behentrimonium chloride, behenamidopropyl dimethylamine and mixtures thereof.

b. Surfactants

In one example, the fibrous element hydration controlling system comprises a surfactant. In one example, the fibrous element hydration controlling system comprises a nonionic surfactant.

Non-limiting examples of nonionic surfactants suitable for use as the fibrous element hydration controlling system include nonionic surfactants selected from the group consisting of: reverse alkyl glucamides, betaines, alkyl glucosides, tertiary amino compounds, alkanolamides and mixtures thereof.

In one example, the fibrous element hydration controlling system comprises an alkanolamide. Non-limiting examples of alkanolamides suitable for use as the fibrous element hydration controlling system include alkanolamides selected from the group consisting of: cocamide DEA, cocamide MEA, cocamide MIPA, isosteararnide DEA, isosteararnide MEA, isostearamide MIPA, lanolinamide DEA, lauramide DEA, lauramide MEA, lauramide MIPA, linoleamide DEA. linoleamide MEA, linoleamide MIPA, myristarnide DEA, myristamide MEA, myristamide MIPA, Oleamide DEA, Oleamide MEA, Oleamide MIPA, palmamide DEA palmarnide MEA, palmarnide MIPA, palmitamide DEA, palmitanide MEA, paln kernelanide DEA, palm kernelamide ME A, palm kernelamide MIPA, peanutamide MEA, peanutamide MIPA, soyamide DEA, stearanide DEA, stearamide MEA, steararnide MIPA, tallamide DEA, tallowamide DEA, tallowamide MEA, undecylenarnide DEA, undecylenanide MEA, PEG-6 cocamide and mixtures thereof.

In one example, the fibrous element hydration controlling system comprises triethanolamine.

c. Salt or Salt-Generating Systems

In one example, the fibrous element hydration controlling system comprises a salt, which may be present in the fibrous elements and/or fibrous element-forming compositions and ultimately in the articles formed therefrom and/or may be an external fibrous element hydration controlling system.

In one example, the fibrous element hydration controlling system comprises a salt-generating system. The salt-generating system may be an effervescent system.

In one example, the fibrous element hydration controlling system comprises an effervescent system. The effervescent system may comprise an acid, such as an effervescent acid, for example an effervescent acid particle, a salt, such as an effervescent salt, for example an effervescent salt particle, and combinations thereof. In one example, the effervescent system comprises one or more particles (in particle form), for example an effervescent acid particle and/or an effervescent salt particle. In one example, the effervescent system comprises an agglomerate comprising an acid, such as an effervescent acid, for example an effervescent acid particle, and a salt, such as an effervescent salt, for example an effervescent salt particle, with or without a binder, such as a surfactant.

In one example, the external fibrous element hydration controlling system comprise an effervescent system. The effervescent system may comprise one or more effervescent acids, for example effervescent acid particles, and one or more effervescent salts, for example effervescent salt particles. In one example, either or both the effervescent acid and the effervescent salt may be in anhydrous form.

Examples of suitable effervescent salts, which may be in particle form, include salts such as alkali and alkali earth metal salts. Non-limiting examples of suitable alkali metal salts include sodium carbonate, calcium carbonate, magnesium carbonate, ammonium carbonate, potassium carbonate, sodium bicarbonate, potassium bicarbonate, calcium bicarbonate, or mixtures thereof. In one example, the effervescent salt particle may exhibit a particle size range from about 50 μm to about 200 μm and/or from about 50 μm to about 150 μm and/or from about 50 μm to about 100 μm.

Examples of suitable effervescent acids, which may be in particle form, include, but are not limited to tartaric acid, citric acid, fumaric acid, succinic acid, adipic acid, malic acid, oxalic acid, or sulfamic acid, either alone or in combination. Polymeric organic acids can also be used; non-limiting examples include poly (acrylic acid), poly (acrylic acid co-maleic acid), poly (maleic acid), and the like. Non-limiting examples of inorganic acids that can be used include urea hydrochloride, sodium bisulfate, phosphoric acid, and the like. In one example, the effervescent acid is citric acid and/or tartaric acid.

In certain aspects, the effervescent acid comprises a coating. The coating can help prevent the effervescent acid from prematurely activating the effervescent salt. Suitable coatings include maltodextrin, citrate, or anti-caking agents such as silicon dioxide. In one example, the effervescent acid comprises citric acid coated with maltodextrin (available under the tradename Citric Acid DC), citric acid coated with citrate (available under the tradename CITROCOAT® N), or citric acid coated with silicon dioxide (available under the tradename Citric Acid S40).

In one example, the effervescent acid may be added in an amount of about 10% to about 60% by weight of the effervescent system and the effervescent salt may be added in an amount of about 10% to 60% by weight of the effervescent system.

In one example, the effervescent acid and/or effervescent salt may comprise from about 0.1% to about 50% and/or from about 1% to about 40% and/or from about 5% to about 30% by weight on a dry fibrous element basis and/or a dry particle basis and/or dry fibrous structure and/or dry article basis.

### Optional Active Agents

In addition to the above-described active agents, the fibrous elements and/or fibrous element-forming compositions and/or articles of the present invention may further comprise one or more optional active agents, which may be in the form of particles, for example active agent-containing particles. The optional active agents may be present in the fibrous elements and/or fibrous element-forming composition and/or external to the fibrous elements, such as commingled, for example as active agent-containing particles, with the fibrous elements prior to forming the article of the present invention and/or applied to a surface of the fibrous elements prior to or after forming the article of the present invention and/or added as layer between two or more layers of fibrous elements during formation of the article of the present invention and/or added between two or more fibrous structures when forming a multi-fibrous structure/multi-ply article of the present invention.

Non-limiting examples of optional active agents are selected from the group consisting of: perfumes, colorants, for example dyes and/or pigments, preservatives, opacifiers, for example titanium dioxide, mica, sunscreen agents including organic and/or inorganic sunscreen agents, for example zinc oxide, titanium dioxide, mica, natural oils, vitamins, for example panthenol, Vitamin E, Vitamin B, Vitamin C, skin conditioning agents, for example petrolatum, mineral oil, silicone, skin soothing agents, for example zinc oxide, petrolatum, sensates, such as cooling agents, for example menthol and menthol lactate, and/or such as warming agents, for example capsaicin and carvacrol, powders, for example hena powder, turmeric powder, antioxidants, rosacea, for example salicylic acid, green mica, azelaic acid, skin benefit agents, for example retinol, hyaluronic acid, humectants, peptides, pyrithiones, for example zinc pyrithione, strobilurins, for example azoxystrobin, piroctone derivatives and/or salts, for example piroctone olamine, and mixtures thereof.

In one example, one or more optional active agents may comprise warming agents, such as encapsulated warming agents and/or active agents that produce an exothermic reaction (release heat), for example when contacted with water, such as zeolites, salts and other reactive ingredients.

In one example, one or more optional active agents may comprise cooling agents, such as encapsulated cooling agents and/or active agents that produce an endothermic reaction (absorbs heat and thus cools its surroundings), for example when contacted with water.

In one example, the optional active agent comprises a vitamin, for example a Vitamin B, such as Vitamin B-3, for example niacinamide, and/or Vitamin E.

In one example, the optional active agent comprises niacinamide.

In one example, the optional active agent comprises panthenol.

In one example, the optional active agent comprises an oil, for example a natural oil, and/or butter, for example shea butter and cocoa butter, which may be in neat form and/or encapsulate form and/or particle form and/or matrix particle form.

The oil may be a natural oil, for example a plant-based oil.

In one example, the oil may be a synthetic oil, for example a hydrocarbon oil and/or a triglyceride oil. In one example, the oil may be a medium chain triglyceride oil (MCT).

Non-limiting examples of oils, for example natural oils, for use in the present invention are selected from the group consisting of: peppermint oil, spearmint oil, argan oil, jojoba oil, coconut oil, palm oil, palm kernel oil, olive oil, soybean oil, flax oil, almond oil, avocado oil, grapeseed oil, rapeseed oil, sunflower oil, safflower oil, terta-hydro curcumin oil and mixtures thereof.

In one example, the optional active agents comprise a vitamin, for example a Vitamin B such as niacinamide, and a natural oil, for example jojoba oil.

In one example, the optional active agents comprise a niacinamide and a natural oil, for example jojoba oil.

In one example, the optional active agents comprise a skin benefit agent, for example retinol, hyaluronic acid, humectants, peptides and mixtures thereof.

In one example, the optional active agents comprise a humectant, for example aloe.

Non-limiting examples of optional active agents include optional active agents that provide moisturization, barrier improvement, anti-fungal, anti-microbial and anti-oxidant, anti-itch, and cooling and/or warming effects. Non-limiting examples of such optional active agents include but are not limited to: Vitamins E and F, aloe, natural extracts/oils including mint, such as peppermint oil, spearmint oil, argan oil, jojoba oil, coconut oil, palm kernel oil, soybean oil, and other plant-based oils, for example oils that come from seeds such as (flax oil, almond oil, etc.) and oils that come from fruits (avocado oil), salicylic acid, niacinamide, caffeine, panthenol, glycols, glycolic acid, lactic acid. PCA, PEGs, erythritol. glycerin, triclosan. lactates, hyaluronates. allantoin and other ureas, betaines, sorbitol, glutamates, xylitols, menthol, menthyl lactate, iso cyclonone, benzyl alcohol, a compound comprising the following structure:

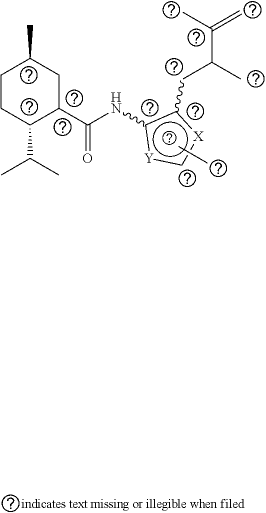

R₁ is selected from H, alkyl, amino alkyl, alkoxy; H₂, O, —OR₁, —N(R₁)₂, OPO(OR₁)ₓ, —PO(OR₁)ₓ, —P(OR₁)ₓ where x=1-2; NR₁, O, —OPO(OR₁)ₓ, —PO(OR₁)ᵣ, —P(OR)ₓ where x=1-2; H₂, O; X, independently selected from H, aryl, naphthyl for n=0; X, aliphatic CH₂ or aromatic CH for n≥1 and Z is selected from aliphatic CH₂, aromatic CH, or heteroatom; lower alkoxy, lower alkylthio, aryl, substituted aryl or fused aryl; and stereochemistry is variable at the positions marked*.

In one example, the optional active agents comprise one or more sensates. Sensates can be materials that provide the sensation of a thermal change, e.g., heating or cooling. Sensates provide skin sensation and comfort benefits. Non-limiting examples of sensates include: p-Methane-3,8-diol; Isopulegol; Menthoxypropane-1,2-diol; Curcumin; Menthyl Lactate; Gingerol; Icilin; Menthol; Tea Tree Oil; Methyl Salicylate; Camphor; Peppermint Oil; N-Ethyl-p-menthane-3-carboxamide; N-[4-(Cyanomethyl)phenyl]-2-isopropyl-5-methylcyclohexane-carboxamide; Ethyl 3-(p-menthane-3-carboxamido)acetate; 2-Isopropyl-N,2,3-trimethylbutyramide; Menthone glycerol ketal; capsaicin, carvacrol, menthol, and mixtures thereof.

Some optional active agents may be considered different classes of optional active agents, for example peppermint oil can be considered an oil, for example a natural oil, as well as a sensate.

In one example, the fibrous elements and//or article may comprise one or more optional active agents, and one or more fibrous element hydration controlling systems, for example an external fibrous element hydration controlling system, such as an effervescent system.

### Printing

The articles of the present invention may further comprise a graphic printed on one of more of its surfaces. A graphic may be printed directly onto the fibrous structure. More particularly, the fibrous structure may include a first surface and a second surface opposite the first surface, and one or more graphics may be printed directly on the first and/or second surfaces of the fibrous structure. In some embodiments, the graphic comprises ink positioned on the first and/or second surface. It is also to be appreciated that the ink may penetrate the fibrous elements, for example inter-entangled fibrous elements, such as filaments, and void areas, for example 100 microns or less into the fibrous structure below the surface on which the ink is applied. As such, the ink may reside on the fibrous structure and/or within the fibrous structure at various depths below the first and/or second surface. In some embodiments, the graphics may be applied such that the fibrous structures have various wet and/or dry ink adhesion ratings. In addition, the graphics may be applied such that the fibrous structure may exhibit desired certain physical properties, such as for example, desired ranges of a geometric mean modulus, geometric mean elongation, and/or geometric means tensile strength.

### Particles

In addition to the fibrous elements, the articles of the present invention may further comprise particles. The particles may be water-soluble or water-insoluble. In one example, one group of particles may be water-soluble and a different group of particles may be water-insoluble. In another example, the particles may comprise one or more active agents and/or one or more optional active agents, such as an oil, (in other words, the particles may comprises active agent-containing particles and/or optional active agent-containing particles). In another example, the particles may comprise one or more fibrous element hydration controlling systems, for example an external fibrous element hydration controlling system, such as an effervescent system. In still another example, the particles may consist essentially of and/or consist of one or more active agents and/or optional active agents (in other words, the particles may comprise 100% or about 100% by weight on a dry particle basis of one or more active agents and/or optional active agents). In still another example, the particles may consist essentially of and/or consist of one or more fibrous element hydration controlling systems, for example external fibrous element hydration controlling systems (in other words, the particles may comprise 100% or about 100% by weight on a dry particle basis of one or more fibrous element hydration controlling systems, for example external fibrous element hydration controlling systems).

In one example, the particles comprise agglomerates of different materials, for example different sub-particles, such as one or more effervescent salt particles, for example sodium bicarbonate, one or more effervescent acid particles, for example citric acid, wherein the particles may be coated with a binder, such as a surfactant.

In one example, the fibrous structure and/or article of the present invention comprises a plurality of particles and a plurality of fibrous elements, for example filaments, at a weight ratio of particles to fibrous elements of from about 3:1 to about 20:1 and/or from about 5:1 to about 15:1 and/or from about 5:1 to about 12:1 and/or from about 7:1 to about 12:1.

a. Matrix Particles

The articles of the present invention may comprise one or more matrix particles. A “matrix particle” comprises one or more matrix materials in the form of a porous structure comprising a plurality of pores and one or more other materials, for example one or more active agents and/or one or more optional active agents, present within at least one of the pores. The matrix particle may be a hollow matrix particle. In another example, the matrix particle may be non-hollow, for example a continuous porous structure (formed by the one or more matrix materials) comprising a plurality of pores wherein at least one other material is present in at least one of the pores. Examples of matrix particles and materials used therein and processes for making such matrix particles are described in U.S. Patent Application Publication No. 20200093711 A1.

In one example, the matrix particles of the present invention are water-soluble. In another example, the matrix particles of the present invention are water-insoluble. In one example a plurality of matrix particles may comprise water-soluble matrix particles and water-insoluble matrix particles.

In one example, the one or more matrix materials are selected for the matrix particle based upon their compatibility with the one or more other materials to be present in the porese.

In one example, when the matrix particle is a water-soluble matrix particle, for example comprises one or more of the other materials, for example a perfume and/or a silicone, the one or more other materials releases from the matrix particle upon the matrix particle contacting a polar solvent, for example water, and/or upon dissolution of a portion or the entire matrix particle and/or one or more, for example all of the matrix materials.

In one example, when the matrix particle is a water-insoluble matrix particle, for example comprises one or more other materials, for example a perfume and/or a silicone, the one or more other materials releases from the matrix particle upon the matrix particle contacting a polar solvent, which swells the matrix particle and facilitates diffusion, for example increases diffusion of the one or more other materials out of the water-insoluble matrix particle. The diffusion of the one or more other materials may not be complete or fast. This ability to diffuse from the matrix particle is aided by the fact that the one or more matrix materials of the matrix particle are not crosslinked with a crosslinking agent, especially in the case of the other materials comprising silicone.

Leakage, in this case diffusion, of the one or more other materials whether the matrix particle is water-soluble or water-insoluble is impacted by the viscosity of the other materials present in the pores. For example, silicone agents exhibit a higher viscosity than the perfume agents and therefore the silicones leak/diffuse from the matrix particles less rapidly and/or less completely than the perfumes, especially when the matrix particles are in a dry state. In light of this fact, in one example a matrix particle comprising one or more silicones as the other materials, for example void or substantially void of perfumes, comprises uncrosslinked matrix materials. And in another example, a matrix particle comprising one or more perfumes as the other materials, for example void or substantially void of silicones, comprises crosslinked matrix materials.

The design of the matrix particles can result in more robust particles, even if there is some fracture of the matrix particles. In the case of a fracture of the matrix particle, only that fractured portion of the matrix particle releases its other materials, such as a perfume, from its pores, the remaining portions of the fractured matrix particle retain their other materials within their pores until exposed to conditions that trigger release and/or are fractured again. This ability of the matrix particles to fracture but have portions of the matrix particle retain their other materials in their pores is advantageous over encapsulated particles, such as core/shell encapsulates wherein fracture of the core/shell encapsulates results in the total loss of the other materials originally encapsulated within the core/shell encapsulates.

In one example, the matrix particle may comprise from about 10-70 wt. % of one or more other materials present within the pores, from about 21-72 wt. % of one or more matrix materials, from about 3-12 wt. % of a crosslinking agent, and from about 1-6 wt. % of a catalyst, by total weight of the matrix particle (not to exceed 100%).

The crosslinking agent, when present, can be present in an amount effective (in the presence of a catalyst) to crosslink the matrix material, for example polysaccharide, such as starch, to an extent effective to provide the matrix particles with desired durability. The amount can be for example at least about 1 wt. % and/or at least about 2 wt. % and/or at least about 3 wt. % and/or at least about 3.80 wt. % and/or at least about 5 wt. % and/or to about 15 wt. % and/or to about 12 wt. % and/or to about 10 wt. % and/or to about 8 wt. % by total weight of the matrix particle.

Non-limiting examples of suitable crosslinking agents may be selected from the group consisting of dimethyldihydroxy urea, dimethyloldihhyrodyethylene urea, dimethylol urea, dihydroxyethylene urea, dimethylolethylene urea, dimethyldihydroxyethylene urea, citric acid, tartaric acid, malic acid, succinic acid, glutaric acid, citraconic acid, itaconic acid, tartrate monosuccinic acid, maleic acid, poly(acrylic acid), poly(methacrylic acid), poly(maleic acid), poly(methylvinylether-co-maleate) copolymer, copolymers of acrylic acid and copolymers of maleic acid, and mixtures thereof.

In addition to the crosslinking agent, the matrix material may further comprise a catalyst in an amount effective to catalyze the crosslinking of the matrix material, for example polysaccharide, such as starch, to an extent effective to provide the matrix particles with desired durability. The amount can be for example at least about 0.1 wt. % and/or at least about 0.5 wt. % and/or at least about 1 wt. % and/or at least about 2 wt. % and/or to about 7 wt. % and/or to about 6 wt. % and/or to about 5 wt. % and/or to about 2.5 wt. % by total weight of the matrix particle.

The catalyst, when present, may be a reducing agent and/or electron donor and may be selected from the group consisting of ammonium chloride, ammonium sulfate, aluminum chloride, magnesium chloride, magnesium nitrate, sodium hypophosphite, and mixtures thereof.

Flow aids, for example silica flow aids may be included in the matrix particles. The silica flow aid may include precipitated silica, fumed silica, hydrophobic silica, and mixtures thereof. However, adding a flow aid, for example a silica flow aid, may prevent and/or inhibit agglomeration of the matrix particles. Therefore, in one example, the matrix particles of the present invention may be free of and/or substantially free of a flow aid, for example a silica flow aid. In another example the matrix particles may comprise less than 1 wt. % and/or less than 0.5 wt. % and/or less than 0.1 wt. % and/or less than 0.05 wt. % flow aid, for example a silica flow aid, by total weight of the matrix particle.

In one example, a matrix particle comprises one or more matrix materials in the form of a porous structure comprising a plurality of pores, wherein one or more other materials are present in at least one of the pores and/or are dispersed throughout the one or more matrix materials, and optionally, wherein at least one of the one or more other materials is released from the matrix particle upon the matrix particle contacting water and/or upon dissolution of a portion or the entire matrix particle and/or one or more, for example all of the matrix materials.

In one example, a matrix particle of the present invention comprises one or more matrix materials, for example uncrosslinked polyvinyl alcohol, and one or more other materials, for example silicone, such as an aminosilicone, for example terminal aminosilicone, present within one or more pores. In one example, such a matrix particle may be agglomerated with other matrix particles, same or different, for example the same, to form an agglomerated particle comprising a plurality of the matrix particles.

In one example, a matrix particle of the present invention comprises one or more matrix materials, for example uncrosslinked polyvinyl alcohol, and one or more other materials, for example perfume. In one example, such a matrix particle may be agglomerated with other matrix particles, same or different, for example the same, to form an agglomerated particle comprising a plurality of the matrix particles.

In one example, at least one of the one or more matrix materials is a water-soluble matrix material. In one example, at least one of the one or more matrix materials is selected from the group consisting of: polyvinyl alcohol, polysaccharides, gums, gelatin, dextrins, polyethylene glycols, gum arabic, larch, pectin, tragacanth, locust bean, guar, alginates, carrageenans, cellulose gums, karaya, and mixtures thereof.

In one example, the matrix particle exhibits a size of less than 500 μm and/or less than 400 μm and/or less than 300 μm and/or less than 200 μm and/or less than 100 μm and/or to about 20 μm and/or to about 30 μm and/or from about 20 μm to about 500 μm and/or from about 20 μm to about 400 μm and/or from about 20 μm to about 300 μm and/or from about 20 μm to about 200 μm and/or from about 20 μm to about 100 μm and/or from about 20 to about 90 μm and/or from about 30 μm to about 80 μm as measured according to the Median Particle Size Test Method described herein.

ai. Matrix Materials

The matrix particle of the present invention may comprise from about 10 wt. % to about 90 wt. % and/or from about 30 wt. % to about 85 wt. % and/or from about 40 wt. % to about 85 wt. % and/or from about 45 wt. % to about 80 wt. % and/or from about 50 wt. % to about 75 wt. % of one or more matrix materials by total weight of the matrix particle.

Non-limiting examples of suitable matrix materials include matrix materials selected from the group consisting of water-soluble polymers, polyvinyl alcohol, polysaccharides, crosslinking agents, catalysts, polyethylene glycols (PEG), starches, gums, gelatin, dextrins, as well as hydrolyzed gums and hydrolyzed gelatin, polyacrylic acid and its copolymers, polyvinylpyrrolidone and its copolymers, for example cationic copolymers thereof, such as polyvinylpyrrolidone/trimethyl amino ethyl methacrylate (PVP/TMAEMA), polyvinylpyrrolidone/dimethyl aminoethyl methacrylate (PVP/DMAEMA) and polyvinylpyrrolidone/dimethyl aminopropylmethyacrylate (PVP/DMAPMA), polyacrylamide and its copolymers, polyoxazoline and its copolymers, such as poly(2-ethyl oxazoline), polyvinylmethyl ether, polyethyleneimine, polymethacrylic acid, N-isopropyl acrylamide, n-n-dimethylacrylamide, other water-soluble acrylic-based polymers, polyvinyloxazolidone, polycaprolactam, polystyrene sulfonate, polyvinyl formamide, polyvinyl amine, and mixtures thereof. Non-limiting examples of suitable starches include gum arabic, larch, pectin, tragacanth, locust bean, guar, alginates such as sodium alginate and propylene glycol alginates, carrageenans, cellulose gums such as carboxymethyl cellulose, and karaya. Some of the suitable matrix materials have melting points and thus can be melted, but for the present invention, in one example, the suitable matrix materials are soluble in a polar solvent, for example water, which results in the matrix materials forming a porous structure comprising a plurality of pores upon removal of the polar solvent, for example water, during the drying process, for example spray drying process. Such a porous structure formed by the matrix materials is not formed upon cooling a melted matrix material. In other words, cooling of a melted matrix material forms a nonporous structure.

In one example, at least one of the one or more matrix materials is selected from the group consisting of: polyvinyl alcohol, polysaccharides, gums, gelatin, dextrins, polyethylene glycols, gum arabic, larch, pectin, tragacanth, locust bean, guar, alginates, carrageenans, cellulose gums, karaya, polyacrylic acid, polyvinylpyrrolidone, polyacrylamide, and mixtures thereof. In one example, at least one of the one or more matrix materials comprises polyvinyl alcohol, for example water-soluble polyvinyl alcohol.

In one example, at least one of the one or more matrix materials comprises a polysaccharide, for example starch.

In one example, at least one of the one or more matrix materials comprises polyethylene glycol in its dissolved form, for example an aqueous solution of polyethylene glycol. Polyethylene glycol in its melted and subsequently cooled form is not within the scope of the present invention.

In another example, a matrix material may include dextrins, for example carboxylated dextrins derived from oxidized starches containing a controlled amount of carboxyl groups. These carboxylated dextrins may be prepared from oxidized cereal starches such as corn, wheat, waxy maize and waxy sorghum starches. Carboxylated dextrins derived from oxidized root starches, such as tapioca and potato starches, may also be used. All of these carboxylated dextrins may be compatible with volatile oils, like perfumes.

The matrix material may comprise polyethylene glycol (PEG). The PEG can have a molecular weight from about 4000 to about 10,000 g/mol and/or from about 6000 to about 9,000 g/mol and/or from about 7000 to about 8000 g/mol. In one example, the PEG may have a molecular weight from about 4000 to about 8000 g/mol. The PEG can be solid at room temperature (about 23° C.) with a melting point of about 60° C. In one example, the matrix particle may comprise PEG 8000.

The matrix material may comprise a polysaccharide, which can be present in an amount effective to provide the desired structural and release properties for the matrix particle, for instance at a level of from at least about 5 wt. % and/or at least about 10 wt. % and/or at least about 21 wt. % and/or at least about 25 wt. % to about 80 wt. % and/or to about 72 wt. % and/or to about 60 wt. % and/or to about 50 wt. % by total weight of the matrix particle. The polysaccharide can be selected from the group consisting of octenyl succinic acid anhydride modified starch including modified corn starch, gum arabic, xanthan gum, gellan gum, pectin gum, konjac gum and carboxyalkyl cellulose, and mixtures thereof.

Aii. Other Materials, for Example Hydrophobic Materials/Hydrophobic Active Agents

The matrix particle of the present invention may comprise from about 10 wt. % to about 90 wt. % and/or from about 15 wt % to about 70 wt % and/or from about 20 wt % to about 50 wt % and/or from about 30 wt % to about 45 wt % of one or more other materials by total weight of the matrix particle.

The other material may be a nonpolar material. The other material may be selected from the group consisting of: perfumes, essential oils, oils, vitamin oils, vegetable oils, silicones, shea butter, cocoa butter, petrolatum, tea tree oil, medium-chain (C₆-C₁₂) triglycerides, and mixtures thereof. In one example, the other material may be a perfume. In another example, the other material may include a perfume in combination with a silicone, such as a terminal aminosilicone and/or polydimethylsilicone, and/or oligomeric vegetable oils. In another example, the other material can include two or more and/or three or more different perfumes.

In one example, at least one of the one or more other materials is selected from the group consisting of: perfumes, essential oils, oils, vitamin oils vegetable oils, silicones, shea butter, cocoa butter, petrolatum, grapeseed oil, sunflower oil, olive oil, argan oil, Vitamin E, and mixtures thereof.

In one example, the other materials may comprise a water-insoluble hydrophobic active agent particle, such as silica, titanium dioxide, and/or sodium hexametaphosphate (commonly referred to as Glass H®), and mixtures thereof.

When the other materials comprises a perfume, the perfume may include perfume compositions comprising perfume materials having a Log P (logarithm of octanol-water partition coefficient) of from about 2 to about 12 and/or from about 2.5 to about 8 and/or from about 2.5 to about 6. In one example, the perfume may exhibit a boiling point of less than about 280° C. and/or from about 50° C. to less than about 280° C. and/or from about 50° C. to less than about 265° C. and/or from about 80° C. to less than about 250° C. In one example, the perfume may exhibit an ODT (odor detection threshold) of less than about 100 parts per billion (ppb) and/or from about 0.00001 ppb to less than about 100 ppb and/or from about 0.00001 ppb to less than about 50 ppb and/or from about 0.00001 ppb to less than about 20 ppb.

A wide variety of natural and synthetic chemical ingredients useful as perfumes and/or perfumery ingredients including but not limited to aldehydes, ketones, esters, and mixtures thereof may be used as one or more other materials, for example as a perfume in the matrix particles of the present invention. Non-limiting examples of essential oils, which can be used as one or more of the hydrophobic active agents, include those obtained from orange oil, lemon oil, thyme, lemongrass, citrus, anise, clove, aniseed, rose extract, lavender, citronella, eucalyptus, peppermint, camphor, sandalwood, cinnamon leaf, cedar, pine oil, musk, patchouli, balsamic essence, and mixtures thereof. Essential oils that exhibit antimicrobial properties may also be used as one or more other materials.

The one or more other materials may include vitamin oils. Non-limiting examples of vitamin oils include fat-soluble vitamin-active materials, pro vitamins, and pure or substantially pure vitamins, both natural and synthetic, or chemical derivatives thereof, crude extractions containing such substances, vitamin A, vitamin D, and vitamin E active materials as well as vitamin K, carotene and the like, or mixtures of such vitamin materials.

Non-limiting examples of vegetable oils, which may be used has other materials, include but are not limited to oils derived from palm, corn, canola, sunflower, safflower, rapeseed, castor, olive, soybean, coconut and the like, in both the unsaturated forms and hydrogenated forms, and mixtures thereof.

In one example, a diluent may be mixed with the other material. The diluent suitable to be mixed with the o may be miscible in the other material, for example in a perfume oil or other oil or silicone and may act to reduce the volatility of the other material, for example a fragrance oil.

Non-limiting examples of diluents may include isopropyl myristate, iso E super, triethyl citrate, vegetable oils, hydrogenated oils, and mixtures thereof.

In one example, the other materials exhibit a particle and/or droplet size of at least 0.02 μm to about 200 μm and/or at least 0.1 μm to about 100 μm and/or from about 0.25 μm to about 100 μm and/or from about 0.5 μm to about 75 μm and/or from about 1 μm to about 50 μm and/or from about 1 μm to about 30 μm and/or from about 2 μm to about 15 μm and/or from about 5 μm to about 10 μm. In one example, the droplet size of the hydrophobic active agents is greater than 5 μm and/or greater than 10 μm and/or greater than 15 μm and/or greater than 20 μm and/or greater than 25 μm and/or less than 100 μm and/or less than 75 μm and/or less than 50 μm and/or less than 40 μm. The droplet size of the other materials can be measured by any suitable method know in the art. For example, the droplet size of the other materials may be measured prior to making the matrix particle for example when the other materials is present as droplets in an emulsion and/or the droplet size of the other materials may be measured by dissolving in water the matrix material of the matrix particle leaving the other material droplets within the water.

### Method for Making Fibrous Element-Forming Composition

The fibrous element-forming composition of the present invention may be made by any suitable process so long as the fibrous element-forming composition is suitable for making the fibrous elements and articles of the present invention.

In one example, one or more active agents, for example one or more active agents, are added (in the absence of free water) to a metal beaker and heated to a temperature sufficient to melt the active agents, for example 80° C. The active agents are melted and optionally agitated until they form a homogeneous fluid.

After melting the active agents, one or more auxiliary ingredients, for example one or more structurants, may be added to the homogeneous fluid of active agents. The auxiliary ingredients, when added, are stirred into the homogeneous fluid of active agents until the auxiliary ingredients are dispersed, for example homogeneously dispersed, in the homogeneous fluid of active agents and/or are homogeneously dissolved within the homogeneous fluid of active agents. This all occurs while maintaining the homogeneous fluid of active agents at a temperature of at least the melting point of the lowest melting point active agent, for example from about 70° C. to about 110° C. and/or from about 80° C. to about 110° C.

The fibrous element-forming composition may then be used to make fibrous elements and/or fibrous structures and/or articles of the present invention.

### Method for Making Fibrous Elements

The fibrous elements of the present invention may be made by any suitable process. A non-limiting example of a suitable process for making the fibrous elements is described below.

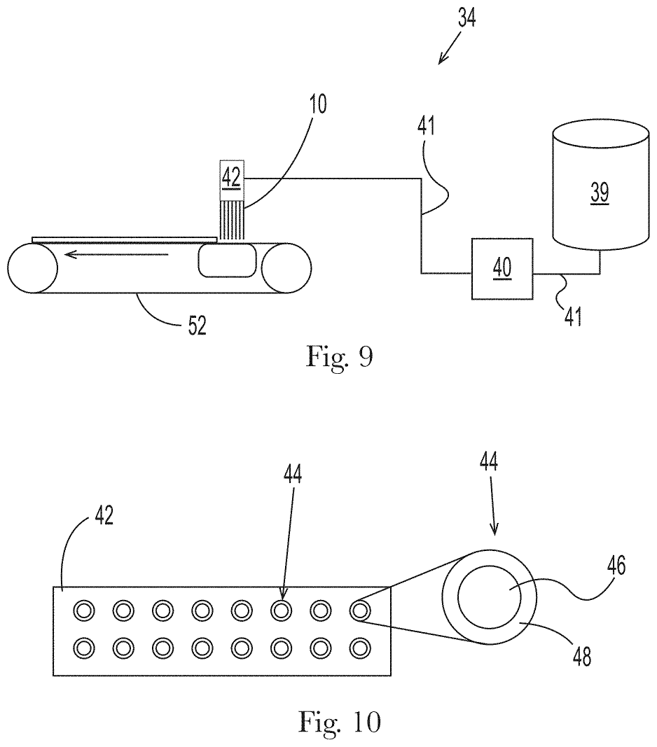

As shown in FIGS. 9 and 10, the fibrous elements 10 of the present invention may be made as follows. Fibrous elements 10 may be formed by means of a small-scale apparatus 34, a schematic representation of which is shown in FIGS. 9 and 10. A pressurized tank 39, suitable for batch operation is filled with a suitable fibrous element-forming composition according to the present invention. A pump 40 such as a Zenith®, type PEP II, having a capacity of 5.0 cubic centimeters per revolution (cm³/rev), manufactured by Parker Hannifin Corporation, Zenith Pumps division, of Sanford, N.C., USA may be used to facilitate transport of the filament-forming composition via pipes 41 to a spinning die 42. The flow of the fibrous element-forming composition from the pressurized tank 39 to the spinning die 42 may be controlled by adjusting the number of revolutions per minute (rpm) of the pump 40. Pipes 41 are used to connect the pressurized tank 39, the pump 40, and the spinning die 42.

The spinning die 42 shown in FIGS. 9 and 10 has several rows of circular extrusion nozzles (fibrous element-forming holes 44) spaced from one another at a pitch P of about 1.524 millimeters (about 0.060 inches). The nozzles have individual inner diameters of about 0.305 millimeters (about 0.012 inches) and individual outside diameters of about 0.813 millimeters (about 0.032 inches). Each individual nozzle is encircled by an annular and divergently flared orifice (concentric attenuation fluid hole 48 to supply attenuation air to each individual melt capillary 46. The fibrous element-forming composition extruded through the nozzles is surrounded and attenuated by generally cylindrical, heated air streams, which may be humidified, supplied through the orifices.

In one example, as shown in FIGS. 9 and 10, a method for making a fibrous element 10 according to the present invention comprises the steps of:

a. providing a fibrous element-forming composition comprising one or more active agents and one or more auxiliary ingredients, such as one or more structurants, and optionally a fibrous element hydration controlling system, for example wherein the fibrous element-forming composition is a melt having a temperature from about 70° C. to about 110° C.; and

b. spinning the fibrous element-forming composition, such as via a spinning die 42, into one or more fibrous elements 10, such as filaments, comprising the one or more active agents and the one or more auxiliary ingredients, and optionally, the fibrous element hydration controlling system.

As shown in FIG. 10, the spinning die 42 may comprise a plurality of fibrous element-forming holes 44 that include a melt capillary 46 encircled by a concentric attenuation fluid hole 48 through which a fluid, such as air, passes to facilitate attenuation of the fibrous element-forming composition into a fibrous element, for example a filament 10 as it exits the fibrous element-forming hole 44.

Attenuation air can be provided by heating compressed air from a source by an electrical-resistance heater, for example, a heater manufactured by Chromalox, Division of Emerson Electric, of Pittsburgh, Pa., USA. An appropriate quantity of steam was added to saturate or nearly saturate the heated air at the conditions in the electrically heated, thermostatically controlled delivery pipe. Condensate is removed in an electrically heated, thermostatically controlled, separator.

The embryonic fibrous elements, for example from the spinning die, which exit the spinning die hot, for example at a temperature of from about 70° C. to about 110° C., are then quenched and cooled by one or more cold air streams, for example cold air streams having a temperature of about from about 5° C. to about 25° C. supplied through cooling nozzles and discharged at an angle of about 900 relative to the general orientation of the embryonic fibrous elements being spun. The cooled embryonic fibrous elements are then collected on a collection device, such as a belt, for example a movable foraminous belt or patterned collection belt to form a fibrous structure and/or article comprising a plurality of the fibrous elements. The fibrous elements may be at a temperature of less than 50° C. and/or less than 45° C. and/or typically, at about 40° C. when they are collected on the collection device. In one example, a plurality of particles of the present invention, for example optional active agent-containing particles and/or external fibrous element hydration controlling systems, may be injected into and/or commingled with the spun fibrous elements prior to collection on the collection device such that the mixture of particles and fibrous elements are collected and form a fibrous structure and/or article. The addition of a vacuum source directly under the collection device where the fibrous elements and optionally, any particles commingled with the fibrous elements are concentrated (formation zone) may be used to aid collection of the fibrous elements.

The fibrous element-forming composition is spun into one or more fibrous elements by any suitable spinning process, such as meltblowing, spunbonding, electro-spinning, and/or rotary spinning. In one example, the fibrous element-forming composition is spun into a plurality of fibrous elements by meltblowing. For example, the fibrous element-forming composition may be pumped from a tank to a meltblown spinnerette. Upon exiting one or more of the fibrous element-forming holes in the spinnerette, the fibrous element-forming composition is attenuated with air to create one or more fibrous elements. The fibrous elements may then be quenched and cooled before collection on a collection device.

### Method for Making Article

In one example, an article of the present invention may be made by the following steps:
a. subjecting one or more active agents to a temperature sufficient to melt the active agents, such as greater than 70° C. to about 110° C. and/or from about 75° C. to about 110° C. and/or from about 75° C. to about 100° C. and/or from about 80° C. to about 95° C. (in the absence of water);
b. adding one or more auxiliary ingredients, for example structurants, to the melted active agents to form a fibrous element-forming composition;
c. adding one or more fibrous element hydration controlling systems to the melted active agents prior to or after adding the one or more auxiliary ingredients;
d. producing, for example by spinning the fibrous element-forming composition, one or more fibrous elements from the fibrous element-forming composition;
e. collecting a plurality of the fibrous elements on a collection device to form a fibrous structure and/or article comprising the plurality of fibrous elements according to the present invention.

In another example of the present invention, a method for making an article of the present invention comprising the following steps:
a. subjecting one or more active agents to a temperature sufficient to melt the active agents;
b. adding one or more auxiliary ingredients, for example structurants, to the melted active agents to form a fibrous element-forming composition;
c. producing a plurality fibrous elements from the fibrous element-forming composition, for example by spinning the fibrous element-forming composition;
d. adding one or more fibrous element hydration controlling systems to the melted active agents prior to or after adding the one or more auxiliary ingredients and/or adding one or more external fibrous element hydration controlling systems to the plurality of fibrous elements formed from the fibrous element-forming composition;
e. collecting a plurality of the fibrous elements, with or with one or more of the external fibrous element hydration controlling systems on a collection device to form a fibrous structure and/or article comprising the plurality of fibrous elements according to the present invention.

In one example, the articles of the present invention may be made by any suitable processes. A non-limiting example of a suitable process for making the articles of the present invention is described below.

A fibrous structure 22 and/or article, for example a fibrous structure layer or ply of the present invention may be made by spinning a fibrous element-forming composition from a spinning die 42, as described in FIGS. 9 and 10, to form a plurality of fibrous elements 10, such as filaments, which are then collected on a collection device, such as a belt 52, for example a patterned collection belt that imparts a texture, such as a three-dimensional texture to at least one surface of the fibrous structure 22 and/or article formed.

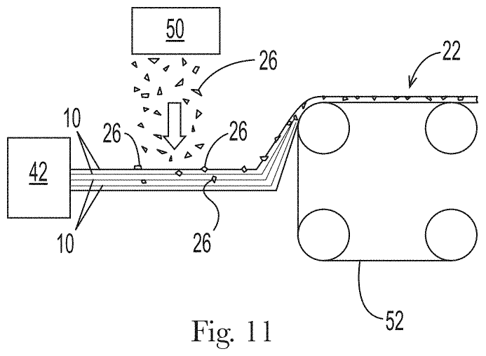

As shown in FIG. 11, a fibrous structure 22 and/or article, for example a fibrous structure layer or ply of the present invention may be made by spinning a fibrous element-forming composition from a spinning die 42, as described in FIGS. 9 and 10, to form a plurality of fibrous elements 10, such as filaments, and then optionally, associating one or more particles 26 provided by a particle source 50, for example a sifter or a airlaid forming head. The particles 26 may be dispersed in the plurality of fibrous elements 10, for example filaments. The mixture of particles 26 and fibrous elements 10 may be collected on a collection device, such as a belt 52, for example a patterned collection belt that imparts a texture, such as a three-dimensional texture to at least one surface of the fibrous structure 22 and/or article formed.

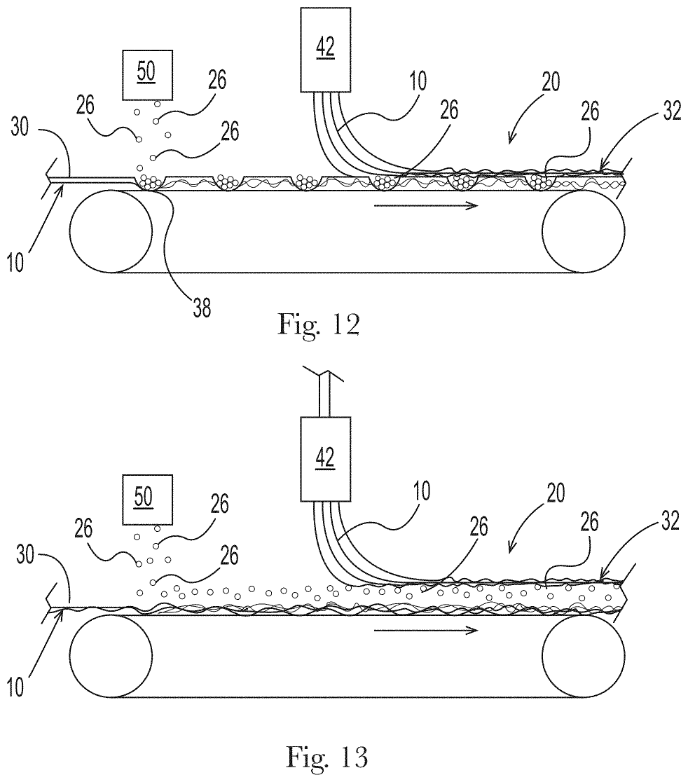

FIG. 12 illustrates an example of a method for making an article 20 according to FIG. 6. The method comprises the steps of forming a first fibrous structure layer 30 of a plurality of fibrous elements 10, for example filaments, such that pockets 28 are formed in a surface of the first fibrous structure layer 30. One or more particles 26 are deposited into the pockets 28 from a particle source 50. A second fibrous structure layer 32 comprising a plurality of fibrous elements 10, for example filaments, produced from a spinning die 42 are then formed on the surface of the first fibrous structure layer 30 such that the particles 26 are entrapped in the pockets 28.

FIG. 13 illustrates yet another example of a method for making an article 20 similar to FIG. 5, but it is multi-layered fibrous structure rather than a multi-ply fibrous structure. The method comprises the steps of forming a first fibrous structure layer 30 of a plurality of fibrous elements 10, for example filaments. One or more particles 26 are deposited onto a surface of the first fibrous structure layer 30 from a particle source 50. A second fibrous structure layer 32 comprising a plurality of fibrous elements 10, for example filaments, produced from a spinning die 42 are then formed on top of the particles 26 such that the particles 26 are positioned between the first fibrous structure layer 30 and the second fibrous structure layer 32.

In one example, in a multi-ply article, one or more fibrous structure plies may be formed and/or deposited directly upon an existing ply of fibrous structure to form a multi-ply fibrous structure. The two or more existing fibrous structure plies may be combined, for example via thermal bonding, gluing, embossing, aperturing, rodding, rotary knife aperturing, die cutting, die punching, needlepunching, knurling, pneumatic forming, hydraulic forming, laser cutting, tufting, and/or other mechanical combining process, with one or more other existing fibrous structure plies to form the multi-ply article of the present invention.

### Printing

As discussed, the fibrous structure and/or article may further include a graphic printed thereon. Printing is generally performed after formation of the fibrous structure and/or article. One or more graphics may be printed on the fibrous structure and/or article by any suitable printing process, for example contact and/or non-contact printing, including ink jet printing.

As previously mentioned, one or more graphics may be printed on a surface of a web and/or fibrous structure and/or article of the present invention. Printing may be characterized as an industrial process in which a graphic is reproduced on a surface. The web and/or fibrous structure and/or article may move relative to the printing station and/or print head. Alternatively and/or in addition, the printing station may also be configured to move relative to the web and/or fibrous structure and/or article while printing. For example, the printing station may move back and forth in lateral directions relative to the web and/or fibrous structure and/or article while printing the graphics.

Various types of printing processes may be used to create the graphics on the fibrous structure and/or article. For example, flexography may be used. In particular, flexography may utilize printing plates made of rubber or plastic with a slightly raised image thereon. The inked plates are rotated on a cylinder which transfers the image to the sheet. Flexography may be a relatively high-speed print process that uses fast-drying inks. Other examples include gravure printing. More particularly, gravure printing utilizes an image etched on the surface of a metal plate. The etched area is filled with ink and the plate is rotated on a cylinder that transfers the image to the sheet. Examples of suitable printing devices are disclosed in U.S. Patent Publication No. 2012/0222576A1.

In one example, a printing station used in the printing process may include a printer in the form of an ink-jet printer. Ink-jet printing is a non-impact dot-matrix printing technology in which droplets of ink are jetted from a small aperture directly to a specified position on a media to create a graphic. Two examples of inkjet technologies include thermal bubble or bubble jet and piezoelectric. Thermal bubble uses heat to apply to the ink, while piezoelectric uses a crystal and an electric charge to apply the ink. In some configurations, the printing station may include a corona treater, which may be positioned upstream of the printer. The corona treater may be configured to increase the surface energy of the surface of the web material to be printed. In some configurations, the printing station may also include an ink curing apparatus. In some configurations, the ink curing apparatus may be in the form of an ultraviolet (UV) light source that may include one or more ultraviolet (UV) lamps, which may be positioned downstream of the printer to help cure inks deposited onto the web material from the printer to form the graphics. In some configurations, the ink curing apparatus may also include an infrared (IR) dryer light source that may include one or more infrared (IR) lamps, which may be positioned downstream of the printer to help dry water-based or solvent-based inks deposited onto the web material from the printer to form the graphics. In some configurations, the ink curing apparatus may include an electron beam (EB or e-beam) generator that may include one or more e-beam electrodes, which may be positioned downstream of the printer to help cure inks deposited onto the web material from the printer to form the graphics.

In addition to the aforementioned various types of printing processes, it is to be appreciated that various types of inks or ink systems may be applied to various types of sheets to create the disclosed patterns, such as solvent-based, water-based, and UV-cured inks. Some embodiments may utilize inks such as Artistri® Inks available from DuPont™, including 500 Series Acid Dye Ink; 5000 Series Pigment Ink; 700 Series Acid Dye Ink; 700 Series Disperse Dye Ink; 700 Series Reactive Dye Ink; 700 Series Pigment Ink; 2500 Series Acid Dye Ink; 2500 Series Disperse Dye Ink; 2500 Series Reactive Dye Ink; 2500 Series Pigment Dye Ink; 3500 Series Disperse Dye Ink; 3500 Series Pigment Dye Ink; and Solar Brite™ Ink. Ink such as disclosed in U.S. Pat. No. 8,137,721 may also be utilized. Water-based inks that may be utilized are available from Environmental Inks and Coatings Corporation, Morganton, N.C., under the following code numbers: EH034677 (yellow); EH057960 (magenta); EH028676 (cyan); EH092391 (black); EH034676 (orange); and EH064447 (green). Some embodiments may utilized water based inks composed of food-grade ingredients and formulated to be printed directly onto ingestible food or drug products, such as Candymark Series inks available in colors such as black pro, red pro, blue pro, and yellow pro, available from Inkcups located in Danvers, Mass. Other broad ranges of general purpose and specialty inks may also be used, including food grade inks available from Videojet Technologies Inc. located in Wood Dale, Ill.

The primary difference among the ink systems is the method used for drying or curing the ink. For example, solvent-based and water-based inks are dried by evaporation, while UV-cured inks are cured by chemical reactions. Inks may also include components, such as solvents, colorants, resins, additives, and (for ultraviolet inks only) UV-curing compounds, that are responsible for various functions. In some embodiments, a multi-stage printing system may be utilized.

To improve ink rub-off resistance, ink compositions used herein may contain a wax. Such waxes may include a polyethylene wax emulsion. Addition of a wax to the ink composition may enhances rub resistance by setting up a barrier which inhibits the physical disruption of the ink film after application of the ink to the fibrous sheet. Based on weight percent solids of the total ink composition, addition ranges for the wax may be from about 0.5% solids to 10% solids. An example polyethylene wax emulsion is JONWAX 26 supplied by S.C. Johnson & Sons, Inc. of Racine, Wis.

### Package

The articles of the present invention may be enclosed in a package, individually wrapped and/or multi-article wrapped. In one example, the package exhibits a moisture barrier with a water vapor transmission rate of less than about 1.0 g H₂O/day/m² and/or less than about 0.5 g H₂O/day/m² and/or less than about 0.3 g H₂O/day/m² and/or about 0.1 g H₂O/day/m².

In one example, the package comprises a container or dispenser that dispenses individual articles during use.

### Method of Use

The present invention also provides for a method of using the articles of the present invention to treat skin and/or hair, such as body hair, for example facial hair and/or leg or other intimate body parts' hair, for example to provide shave prep conditions and/or skin and/or hair benefits. In one example, a method of treating skin and/or hair, comprises the step of contacting a plurality of fibrous elements and/or a fibrous structure and/or article according to the present invention with a fluid, such as water, for example while a user holds the fibrous elements and/or fibrous structure and/or article in a hand or surrogate for a hand. Once wetted, a user then rubs the wetted fibrous elements and/or fibrous structure and/or article in the hand to create a cream. The user then applies the cream to the user's skin and/or hair to be treated. The user then may shave the skin and/or hair, such as with a razor/razor blade to remove hair.

In one example, plurality of fibrous elements and/or fibrous structure and/or article of the present invention is contacted with water, for example water at a temperature of greater than 15° C. and/or greater than 20° C. and/or greater than 25° C. and/or greater than 30° C. and/or greater than 35° C. and/or greater than 40° C. and/or less than 65° C. and/or less than 60° C. and/or less than 55° C. and/or less than 50° C., and the wetting of the fibrous elements and/or fibrous structure and/or article includes hydrating the fibrous elements and/or fibrous structure and/or article. The user may wait for a short period time, for example a few seconds, after contacting the water to the fibrous elements and/or fibrous structure and/or article to allow for hydration prior to rubbing the wetted fibrous elements and/or fibrous structure and/or article. Hydration-time for the wetted fibrous elements and/or fibrous structure and/or article may vary with the fibrous element-forming composition and structure of the fibrous elements within the fibrous structure and/or article, but is generally in the range of 2-20 seconds. In one example, the hydration-time is from about 2 to about 20 seconds and/or from about 5 to about 15 seconds and/or from about 5 to about 10 seconds. In one example, hydration-times longer than about 40 seconds and/or longer than 30 seconds and/or longer than 20 seconds may provide an undesirable consumer experience. In one example, hydration-times shorter than about 1 second and/or about 2 seconds may provide an undesirable consumer experience.

In one example, the article of the present invention is suitable for a single use, in other words, the article is a consumable, single-use article, since it is designed such that the article transforms from a solid shape to a flowable state, such as a cream, and then ultimately disappear after applying to the skin and/or hair and removing, for example by shaving, and then rinsing down a drain with water, for example excess water.

In one example, the fibrous elements and/or fibrous element-forming composition and/or fibrous structure and/or article of the present invention comprises one or more fibrous element hydration controlling systems and/or external hydration controlling systems that facilitates easier removal, for example by rinsing with a liquid, such as water, of a cream resulting from the water-insoluble fibrous elements and/or water-insoluble fibrous structure and/or water-insoluble article from skin and/or hair and/or a razor, such as a razor blade. Without wishing to be bound by theory, it is believed that the fibrous element hydration controlling system and/or external fibrous element hydration controlling system prevents and/or inhibits and/or mitigates and/or reduces and/or delays the hydration (and thus the swelling) of the water-insoluble fibrous elements and/or water-insoluble fibrous structure and/or water-insoluble article sufficiently to permit the creation of a cream that avoids the negatives associated with water-insoluble fibrous elements and/or water-insoluble fibrous structures and/or water-insoluble articles that do not include fibrous element hydration controlling systems and/or external fibrous element hydration controlling systems of the present invention. Water-insoluble fibrous elements and/or water-insoluble fibrous structures and/or water-insoluble articles comprising such water-insoluble fibrous elements and/or water-insoluble fibrous structures and/or water-insoluble articles without fibrous element hydration controlling systems and/or external fibrous element hydration controlling systems hydrate too much resulting in too undesirable swelling at least for shave prep applications, which creates a shave prep cream in which the water-insoluble fibrous elements break apart and spread undesirably causing rinse off negatives when a user attempts to rinse of the user's skin and/or hair and/or razor, for example razor blade.

In one example, the fibrous elements and/or fibrous structures and/or articles of the present invention are dry, for example dry-to-the-touch, that disappears and/or is entirely consumed during use. “Dry-to-the-touch” as used herein means an article is substantially free of liquids, for example water, such that it does not feel damp or wet prior to being contacted with water or other liquids.

In other words, a dry-to-the-touch article of the present invention does not contain liquids, such as water. In one non-limiting example, a dry-to-the-touch article has a water content of less than about 20% and/or less than about 15% and/or less than about 10% and/or less than about 5% and/or less than about 3% and/or less than about 1% and/or about 0% as measured according to the Water Content Test Method described herein.

While not wishing to be bound by theory, the inventors have surprisingly found the articles of the present invention provide consumers with a consumable, single-use article delivering a combination of 1) disappearing and/or being entirely consumed, 2) being dry-to-the-touch, and 3) leaving behind no visible residue on the treated surface. The inventors have also discovered articles of the present invention may also be designed so as they are shippable in efficient e-commerce friendly configurations.

### Non-Limiting Examples

Non-limiting examples of articles made from the fibrous element-forming compositions of the present invention as shown in Table 1 below are made as follows:
a. adding one or more active agents to a metal beaker;
b. heating, for example to 80° C., the metal beaker with stirring/agitation, as needed, until a homogeneous fluid of active agents is formed;
c. maintaining the metal beaker at 80° C.; and
d. adding one or more auxiliary ingredient (such as a structurant) to the homogeneous fluid of active agents with stirring/agitation, as needed, until the auxiliary ingredients are homogeneously dispersed and/or homogeneously dissolved in the homogeneous fluid of active agents resulting in a fibrous element-forming composition that is ready for producing, for example by spinning, water-insoluble fibrous elements that once collected on a collection device form a water-insoluble fibrous structure and/or a water-insoluble article; and
e. optionally adding one or more fibrous element hydration controlling systems to the homogeneous fluid of active agents concurrent with the addition of the active agent and/or prior to and/or concurrent with and/or after the addition of the auxiliary ingredients to the homogeneous fluid of active agents; and
f. optionally adding one or more external fibrous element hydration controlling systems to the water-insoluble fibrous elements before collection of the water-insoluble fibrous elements on a collection device and/or after formation of a water-insoluble fibrous structure and/or water-insoluble article from the water-insoluble fibrous elements; and
g. optionally adding optional active agents, such as oils, vitamins, perfumes and other optional active agents anywhere in the process.

TABLE 1

| | Comparative | | | |
|---|---|---|---|---|
| | Example | | | |
| | (U.S. | | | |
| | Pat. No. | Ex- | Ex- | Ex- |
| | 10,975,339 | ample | ample | ample |
| | Example 7) | 1 | 2 | 3 |
| Ingredient | wt % | wt % | wt % | wt % |
| Fibrous Element-Forming | | | | |
| Composition: | | | | |
| Cetyl Alcohol (Active Agent) | 18% | 14% | 17% | 19% |
| Stearyl Alcohol (Active Agent) | 44% | 36% | 40% | 45% |
| Cocamide MEA (CMEA) | — | — | 10% | 3% |
| (Active Agent/Fibrous Element | | | | |
| Hydration Controlling System) | | | | |
| Lauroyl/Myristoyl Methyl | 9% | 7% | — | — |
| Glucamide (Active Agent) | | | | |
| Behentrimonium Methosulfate | 25% | 20% | 10% | 10% |
| (Active Agent/Fibrous Element | | | | |
| Hydration Controlling System) | | | | |
| Behentrimonium Chloride | — | — | — | — |
| (Active Agent/Fibrous Element | | | | |
| Hydration Controlling System) | | | | |
| PVP K90 (Auxiliary | — | 5% | 6% | 6% |
| Ingredient/Structurant) | | | | |
| PVP K30 (Auxiliary | — | 18% | 17% | 17% |
| Ingredient/Structurant) | | | | |
| PVP K120 (Auxiliary | 4% | — | — | — |
| Ingredient/Structurant) | | | | |
| Total Check | 100% | 100% | 100% | 100% |
| Agglomerate Composition: | | | | |
| Effervescent System (citric | — | 15% | — | — |
| acid/sodium bicarbonate - | | | | |
| 2.5%/2.5%) (Active | | | | |
| Agent/External Fibrous | | | | |
| Element Hydration Controlling | | | | |
| System) - Add-on Level | | | | |
| Agglomerate:Fibrous Element | — | 0.18 | — | — |
| Weight Ratio | | | | |
| | Ex- | Ex- | Ex- | Ex- |
| | ample | ample | ample | ample |
| | 4 | 5 | 6 | 7 |
| Ingredient | wt % | wt % | wt % | wt % |
| Fibrous Element-Forming | | | | |
| Composition: | | | | |
| Cetyl Alcohol (Active Agent) | 17% | 19% | 18% | 18% |
| Stearyl Alcohol (Active Agent) | 41% | 45% | 44% | 44% |
| Cocamide MEA (CMEA) | 10% | 3% | — | — |
| (Active Agent/Fibrous Element | | | | |
| Hydration Controlling System) | | | | |
| Glucotain Clean (Active Agent) | — | — | 9% | 9% |
| Behentrimonium Chloride | 10% | 10% | 25% | 25% |
| (Active Agent/Fibrous Element | | | | |
| Hydration Controlling System) | | | | |
| PVP K90 (Auxiliary | 5% | 5% | — | — |
| Ingredient/Structurant) | | | | |
| PVP K60 (Auxiliary | — | — | — | — |
| Ingredient/Structurant) | | | | |
| PVP K30 (Auxiliary | 17% | 18% | — | — |
| Ingredient/Structurant) | | | | |
| PVP K120 (Auxiliary | — | — | 4% | 4% |
| Ingredient/Structurant) | | | | |
| Total Check | 100% | 100% | 100% | 100% |
| Agglomerate Composition: | | | | |
| Particle 1:Particle 2 | | | | |
| Particle 1: | — | — | | |
| Perfume - Add-on Level | | | 5% | 5% |
| Menthol - Add-on Level | | | 1% | 1% |
| Starch - Add-on Level | | | 9% | 9% |
| Particle 2: | — | — | | |
| Zinc Oxide - Add-on Level | | | | 12% |
| Agglomerate:Fibrous Element | — | — | 0.19 | 0.39 |
| Weight Ratio | | | | |

### Comparative Example

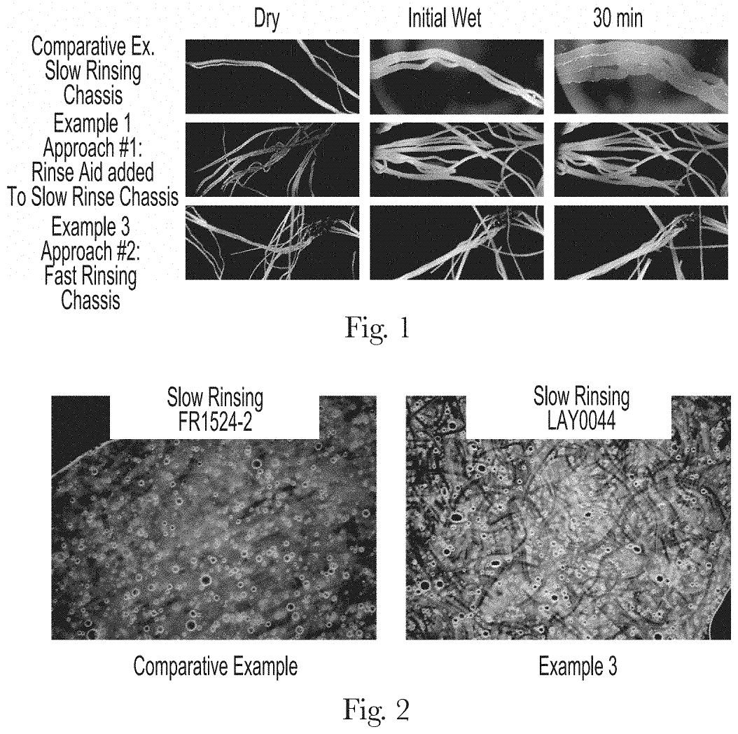

The Comparative Example is made according to U.S. Pat. No. 10,975,339 Example 7. As shown in FIGS. 1 and 2, the water-insoluble article made from the fibrous element-forming composition exhibits undesirable hydration of the water-insoluble fibrous elements and results in undesirable swelling and associated negatives when used for shave prep applications.

The water-insoluble fibrous elements and water-insoluble article of this Comparative Example is made as described in U.S. Pat. No. 10,975,339.

Three plies of the fibrous structure made according to this Comparative Example are stacked and then combined and bonded together via a rod-to-rod rodding solid state formation process having a DOE of 100 to form a multi-ply fibrous structure.

The multi-ply fibrous structure is then die cut into 3.9 cm×3.9 cm rounded squares to form articles having an average weight of 1.24 g+/−0.04 g per article and an average caliper of 2.99 mm per article.

### INVENTIVE EXAMPLES

### Example 1

As shown in FIGS. 1 and 2, the water-insoluble article made from the fibrous element-forming composition of Example 1 exhibits acceptable hydration of the water-insoluble fibrous elements and results in little to no swelling of the water-insoluble fibrous elements and thus avoids the negatives associated with the water-insoluble fibrous elements and water-insoluble article of the Comparative Example.

The water-insoluble fibrous elements and water-insoluble article of Example 1 is made as described hereinabove and below.

Three plies of the fibrous structure made according to this Comparative Example are stacked and then combined and bonded together via a rod-to-rod rodding solid state formation process having a DOE of 125 to form a multi-ply fibrous structure.

The multi-ply fibrous structure is then die cut into 3.9 cm×3.9 cm rounded squares to form articles having an average weight of 1.29 g+/−0.04 g per article and an average caliper of 3.22 mm per article.

### Example 2

The water-insoluble article made from the fibrous element-forming composition of Example 2 exhibits acceptable hydration (similar to or better than Example 1) of the water-insoluble fibrous elements and results in little to no swelling of the water-insoluble fibrous elements and thus avoids the negatives associated with the water-insoluble fibrous elements and water-insoluble article of the Comparative Example.

The water-insoluble fibrous elements and water-insoluble article of Example 2 is made as described hereinabove and below.

Three plies of the fibrous structure made according to this Comparative Example are stacked and then combined and bonded together via a rod-to-rod rodding solid state formation process having a DOE of 150 to form a multi-ply fibrous structure.

The multi-ply fibrous structure is then die cut into 3.9 cm×3.9 cm rounded squares to form articles having an average weight of 1.19 g+/−0.04 g per article and an average caliper of 3.19 mm per article.

### Example 3

As shown in FIGS. 1 and 2, the water-insoluble article made from the fibrous element-forming composition of Example 3 exhibits acceptable hydration (similar to or better than Example 1) of the water-insoluble fibrous elements and results in little to no swelling of the water-insoluble fibrous elements and thus avoids the negatives associated with the water-insoluble fibrous elements and water-insoluble article of the Comparative Example.

The water-insoluble fibrous elements and water-insoluble article of Example 3 is made as described hereinabove and below.

Three plies of the fibrous structure made according to this Comparative Example are stacked and then combined and bonded together via a rod-to-rod rodding solid state formation process having a DOE of 175 to form a multi-ply fibrous structure.

The multi-ply fibrous structure is then die cut into 3.9 cm×3.9 cm rounded squares to form articles having an average weight of 1.22 g+/−0.04 g per article and an average caliper of 3.48 mm per article.

### Example 4

The water-insoluble article made from the fibrous element-forming composition of Example 4 exhibits acceptable hydration (similar to or better than Example 1) of the water-insoluble fibrous elements and results in little to no swelling of the water-insoluble fibrous elements and thus avoids the negatives associated with the water-insoluble fibrous elements and water-insoluble article of the Comparative Example.

The water-insoluble fibrous elements and water-insoluble article of Example 4 is made as described hereinabove and below.

Three plies of the fibrous structure made according to this Comparative Example are stacked and then combined and bonded together via a rod-to-rod rodding solid state formation process having a DOE of from about 100 to 175 to form a multi-ply fibrous structure.

The multi-ply fibrous structure is then die cut into 3.9 cm×3.9 cm rounded squares to form articles having an average weight of from about 1.00 g to about 1.50 g per article and an average caliper of from about 2.50 mm to about 4.00 mm per article.

### Example 5

The water-insoluble article made from the fibrous element-forming composition of Example 5 exhibits acceptable hydration (similar to or better than Example 1) of the water-insoluble fibrous elements and results in little to no swelling of the water-insoluble fibrous elements and thus avoids the negatives associated with the water-insoluble fibrous elements and water-insoluble article of the Comparative Example.

The water-insoluble fibrous elements and water-insoluble article of Example 5 is made as described hereinabove and below.

Three plies of the fibrous structure made according to this Comparative Example are stacked and then combined and bonded together via a rod-to-rod rodding solid state formation process having a DOE of from about 100 to 175 to form a multi-ply fibrous structure.

The multi-ply fibrous structure is then die cut into 3.9 cm×3.9 cm rounded squares to form articles having an average weight of from about 1.00 g to about 1.50 g per article and an average caliper of from about 2.50 mm to about 4.00 mm per article.

### Test Methods

Unless otherwise specified, all tests described herein including those described under the Definitions section and the following test methods are conducted on samples that have been conditioned in a conditioned room at a temperature of 23° C.±1.0° C. and a relative humidity of 50%±2% for a minimum of 2 hours prior to the test. The samples tested are “usable units.” “Usable units” as used herein means fibrous structures and/or articles. All tests are conducted under the same environmental conditions and in such conditioned room. Do not test samples that have defects such as wrinkles, tears, holes, and like. Samples conditioned as described herein are considered dry samples (such as “dry filaments”) for testing purposes. All instruments are calibrated according to manufacturer's specifications.

### Basis Weight Test Method

The basis weight of an article is calculated by dividing the article mass by the projected area of the article as viewed orthogonally to the plane of the article length and width. The article basis weight is reported to the nearest 0.01 g/m².

### Tensile Test Method: Elongation, Tensile Strength, TEA and Modulus

Elongation, Tensile Strength, TEA and Tangent Modulus are measured on a constant rate of extension tensile tester with computer interface (a suitable instrument is the EJA Vantage from the Thwing-Albert Instrument Co. West Berlin, N.J.) using a load cell for which the forces measured are within 10% to 90% of the limit of the cell. Both the movable (upper) and stationary (lower) pneumatic jaws are fitted with smooth stainless steel faced grips, 25.4 mm in height and wider than the width of the test specimen. An air pressure of about 60 psi is supplied to the jaws.

Eight usable units of a fibrous structure, or article sheet are divided into two stacks of four samples each. The samples in each stack are consistently oriented with respect to machine direction (MD) and cross direction (CD). One of the stacks is designated for testing in the MD and the other for CD. Using a one inch precision cutter (Thwing Albert JDC-1-10, or similar) cut 4 MD strips from one stack, and 4 CD strips from the other, with dimensions of 1.00 in ±0.01 in wide by 3.0-4.0 in long. Each strip of one usable unit thick will be treated as a unitary specimen for testing.

Program the tensile tester to perform an extension test, collecting force and extension data at an acquisition rate of 20 Hz as the crosshead raises at a rate of 2.00 in/min (5.08 cm/min) until the specimen breaks. The break sensitivity is set to 80%, i.e., the test is terminated when the measured force drops to 20% of the maximum peak force, after which the crosshead is returned to its original position.

Set the gauge length to 1.00 inch. Zero the crosshead and load cell. Insert at least 1.0 in of the unitary specimen into the upper grip, aligning it vertically within the upper and lower jaws and close the upper grips. Insert the unitary specimen into the lower grips and close. The unitary specimen should be under enough tension to eliminate any slack, but less than 5.0 g of force on the load cell. Start the tensile tester and data collection. Repeat testing in like fashion for all four CD and four MD unitary specimens. Program the software to calculate the following from the constructed force (g) verses extension (in) curve:

Tensile Strength is the maximum peak force (g) divided by the sample width (in) and reported as g/in to the nearest 1 g/in.

Adjusted Gauge Length is calculated as the extension measured at 3.0 g of force (in) added to the original gauge length (in).

Elongation is calculated as the extension at maximum peak force (in) divided by the Adjusted Gauge Length (in) multiplied by 100 and reported as % to the nearest 0.1%

Total Energy (TEA) is calculated as the area under the force curve integrated from zero extension to the extension at the maximum peak force (g*in), divided by the product of the adjusted Gauge Length (in) and specimen width (in) and is reported out to the nearest 1 g*in/in².

Replot the force (g) verses extension (in) curve as a force (g) verses strain curve. Strain is herein defined as the extension (in) divided by the Adjusted Gauge Length (in). Program the software to calculate the following from the constructed force (g) verses strain curve:

Tangent Modulus is calculated as the slope of the linear line drawn between the two data points on the force (g) versus strain curve, where one of the data points used is the first data point recorded after 28 g force, and the other data point used is the first data point recorded after 48 g force. This slope is then divided by the specimen width (2.54 cm) and reported to the nearest 1 g/cm.

The Tensile Strength (g/n), Elongation (%), Total Energy (g*in/in²) and Tangent Modulus (g/cm) are calculated for the four CD unitary specimens and the four MD unitary specimens. Calculate an average for each parameter separately for the CD and MD specimens.

### Calculations:

Geometric Mean Tensile=Square Root of [MD Tensile Strength (g/in)×CD Tensile Strength (g/in)]

Geometric Mean Peak Elongation=Square Root of [MD Elongation (%)×CD Elongation (%)]

Geometric Mean TEA=Square Root of [MD TEA (g*in/in²)×CD TEA (g/in²)]

Geometric Mean Modulus=Square Root of [MD Modulus (g/cm)×CD Modulus (g/cm)]

Total Dry Tensile Strength (TDT)=MD Tensile Strength (g/in)+CD Tensile Strength (g/in)

Total TEA=MD TEA (g*in/in²)+CD TEA (g*in/in²)

Total Modulus=MD Modulus (g/cm)+CD Modulus (g/cm)

Tensile Ratio=MD Tensile Strength (g/in)/CD Tensile Strength (g/in)

### Water Content Test Method

The water (moisture) content present in an article is measured using the following Water Content Test Method. An article sample, or portion thereof, is placed in a conditioned room at a temperature of 23° C.±1.0 C° and a relative humidity of 50%±2% for at least 24 hours prior to testing. Under the temperature and humidity conditions mentioned above, using a balance with at least four decimal places, the weight of the sample is recorded every five minutes until a change of less than 0.5% of previous weight is detected during a 10 minute period. The final weight is recorded as the “equilibrium weight”. Within 10 minutes, the samples are placed into a forced air oven on top of foil, or inside an aluminum tray for 24 hours at 70° C.±2 C.° at a relative humidity of 4%±2% for drying. After the 24 hours of drying, the sample is removed and weighed within 15 seconds. This weight is designated as the “dry weight” of the sample. The water (moisture) content of the sample is calculated according to the following equation:

$$\% Water Content = \frac{Equilibrium Weight - Dry Weight}{Dry Weight} ⨯ 100$$

The % Water Content is measured for 3 replicate samples, and averaged to give the reported to the nearest 0.1%.

### Median Particle Size Test Method

This test method must be used to determine median particle size.

The median particle size test is conducted to determine the median particle size of the seed material using ASTM D 502-89, “Standard Test Method for Particle Size of Soaps and Other Detergents”, approved May 26, 1989, with a further specification for sieve sizes used in the analysis. Following section 7, “Procedure using machine-sieving method,” a nest of clean dry sieves containing U.S. Standard (ASTM E 11) sieves #8 (2360 um), #12 (1700 um), #16 (1180 um), #20 (850 um), #30 (600 um), #40 (425 um), #50 (300 um), #70 (212 um), #100 (150 um) is required. The prescribed Machine-Sieving Method is used with the above sieve nest. The seed material is used as the sample. A suitable sieve-shaking machine can be obtained from W.S. Tyler Company of Mentor, Ohio, U.S.A.

The data are plotted on a semi-log plot with the micron size opening of each sieve plotted against the logarithmic abscissa and the cumulative mass percent (Q₃) plotted against the linear ordinate. An example of the above data representation is given in ISO 9276-1:1998, “Representation of results of particle size analysis —Part 1: Graphical Representation”, Figure A.4.

The seed material median particle size (D₅₀), for the purpose of the present disclosure, is defined as the abscissa value at the point where the cumulative mass percent is equal to 50 percent, and is calculated by a straight line interpolation between the data points directly above (a50) and below (b50) the 50% value using the following equation:

D₅₀=10\hat{ }[Log(Dₐ₅₀)−(Log(Dₐ₅₀)−Log(D_{b50}))*(Qₐ₅₀−50%)/(Qₐ₅₀−Q_{b50})]

where Qₐ₅₀ and Q_{b50} are the cumulative mass percentile values of the data immediately above and below the 50th percentile, respectively; and Dₐ₅₀ and D_{b50} are the micron sieve size values corresponding to these data.

In the event that the 50^{th} percentile value falls below the finest sieve size (150 um) or above the coarsest sieve size (2360 um), then additional sieves must be added to the nest following a geometric progression of not greater than 1.5, until the median falls between two measured sieve sizes.

The Distribution Span of the Seed Material is a measure of the breadth of the seed size distribution about the median. It is calculated according to the following:

Span=(D₈₄/D₅₀+D₅₀/D₁₆)/2
Where D₅₀ is the median particle size and D₈₄ and D₁₆ are the particle sizes at the sixteenth and eighty-fourth percentiles on the cumulative mass percent retained plot, respectively.

In the event that the D₁₆ value falls below the finest sieve size (150 um), then the span is calculated according to the following:

Span=(D₈₄/D₅₀).

In the event that the D₈₄ value falls above the coarsest sieve size (2360 um), then the span is calculated according to the following:

Span=(D₅o/D₁₆).

In the event that the D₁₆ value falls below the finest sieve size (150 um) and the D₈₄ value falls above the coarsest sieve size (2360 um), then the distribution span is taken to be a maximum value of 5.7.

### Diameter Test Method

The diameter of a discrete fibrous element or a fibrous element within a fibrous structure is determined by using a Scanning Electron Microscope (SEM) or an Optical Microscope and an image analysis software. A magnification of 200 to 10,000 times is chosen such that the fibrous elements are suitably enlarged for measurement. When using the SEM, the samples are sputtered with gold or a palladium compound to avoid electric charging and vibrations of the fibrous element in the electron beam. A manual procedure for determining the fibrous element diameters is used from the image (on monitor screen) taken with the SEM or the optical microscope. Using a mouse and a cursor tool, the edge of a randomly selected fibrous element is sought and then measured across its width (i.e., perpendicular to fibrous element direction at that point) to the other edge of the fibrous element. A scaled and calibrated image analysis tool provides the scaling to get actual reading in μm. For fibrous elements within a fibrous structure, several fibrous elements are randomly selected across the sample of the fibrous structure using the SEM or the optical microscope. At least two portions of the fibrous structure are cut and tested in this manner. Altogether at least 100 such measurements are made and then all data are recorded for statistical analysis. The recorded data are used to calculate average (mean) of the fibrous element diameters, standard deviation of the fibrous element diameters, and median of the fibrous element diameters.

Another useful statistic is the calculation of the amount of the population of fibrous elements that is below a certain upper limit. To determine this statistic, the software is programmed to count how many results of the fibrous element diameters are below an upper limit and that count (divided by total number of data and multiplied by 100%) is reported in percent as percent below the upper limit, such as percent below 1 micrometer diameter or %-submicron, for example. We denote the measured diameter (in μm) of an individual circular fibrous element as di.

In the case that the fibrous elements have non-circular cross-sections, the measurement of the fibrous element diameter is determined as and set equal to the hydraulic diameter which is four times the cross-sectional area of the fibrous element divided by the perimeter of the cross-section of the fibrous element (outer perimeter in case of hollow fibrous elements). The number-average diameter, alternatively average diameter is calculated as:

$${d}_{num} = \frac{{\sum }_{i = 1}^{n} {d}_{i}}{n}$$

### Lamellar Structure Test Method

The Lamellar Structure Test Method makes use of small-angle x-ray scattering (SAXS) to determine if a lamellar structure is present in an article either in a conditioned, dry state or upon wetting after having been previously in a conditioned, dry state. Fibrous material articles are conditioned at a temperature of 23° C.±2.0° C. and a relative humidity of 40%±10% for a minimum of 12 hours prior to the test. Articles conditioned as described herein are considered to be in a conditioned, dry state for the purposes of this invention. All instruments are calibrated according to manufacturer's specifications.

Dry Sample Preparation

To prepare a sample to be analyzed directly in the conditioned, dry state, a specimen of about 1.0 cm diameter disc is isolated from the center of an article and is loaded into a conventional SAXS solid sample holder with aperture diameter between 4 and 5 mm. Multiple specimen discs may be extracted from multiple articles and stacked, if necessary, to ensure sufficient scattering cross-section. The loaded sample holder is immediately placed in the appropriate instrument for data collection.

Wet Sample Preparation

Three samples are analyzed upon wetting from the dry, conditioned state. Specimens are extracted from dry, conditioned articles and hydrated with water in order to achieve three separate preparations each possessing a different specimen-to-water mass ratio. The three different specimen-to-water mass ratios to be prepared are 1:5, 1:9, and 1:20. For each mass ratio, one or more specimens (as needed) 1 cm in diameter are extracted from the geometric centers of one or more articles in the dry, conditioned state are hydrated with 23° C.±2.0° C. filtered deionized (DI) water in order to achieve the intended specimen-to-water mass ratio. Each of the three specimen/water mixtures (each corresponding to a different mass ratio) is stirred under low shear gently by hand at room temperature using a spatula until visibly homogenous. Each specimen/water mixture is then immediately loaded into a separate quartz capillary tube with outer diameter 2.0 mm in diameter and 0.01 mm wall thickness. The capillary tubes are immediately sealed with a sealant such as an epoxy resin to prevent the evaporation of water from the preparations. The sealant is permitted to dry for at least 2 hours and until dry at a temperature of 23° C.±2.0° C. prior to sample analysis. Each prepared wet sample is introduced into an appropriate SAXS instrument and data are collected.

Testing and Analysis

Samples are tested using SAXS in 2-dimension (2D) transmission mode over an angular range in of 0.3° to 3.0° 2θ, to observe the presence and spacing of any intensity bands in the x-ray scattering pattern. The test is conducted using a SAXS instrument (such as the NanoSTAR, Bruker AXS Inc., Madison, Wis., U.S.A., or equivalent). Conditioned, dry samples are analyzed under ambient pressure. Sealed liquid samples are analyzed in the instrument under vacuum. All samples are analyzed at a temperature of 23° C.±2.0° C. The x-ray tube of the instrument is operated at sufficient power to ensure that any scattering bands present are clearly detected. The beam diameter is 550±50 m. One suitable set of operating conditions includes the following selections: NanoSTAR instrument; micro-focus Cu x-ray tube using the Kα line at 1.54 Å; 45 kV and 0.650 mA power; Vantec2K 2-Dimensional area detector; collection time of 1200 seconds; and distance between the sample and detector of 112.050 cm. The raw 2-D SAXS scattering pattern is integrated azimuthally to determine intensity (I) as a function of the scattering vector (q), which are expressed throughout this method units of reciprocal angstroms (Å⁻¹). The values for q are calculated by the SAXS instrument according to the following equation:

$$q = \frac{4 π}{λ} sin θ$$

where:

2θ is the scattering angle; and

λ is the wavelength used.

For each integrated SAXS analyzed, the value of q in Å⁻¹ corresponding to each intensity peak on the plot of I vs q is identified and recorded from smallest to largest. (One of skill in the art knows that a sharp peak in q near the origin corresponds to scatter off of the beam stop and is disregarded in this method.) The value of q corresponding to the first intensity peak (the lowest value of q) is referred to as q*.

For a sample corresponding to a specimen (taken from a fibrous material article) analyzed directly in the dry, conditioned state, if an intensity peak is present at 2q*±0.002 Å⁻¹, then the fibrous material of which the article is composed is determined to exhibit a lamellar structure, and the characteristic d-spacing parameter is defined as 2π/q*. If no intensity peak is present at 2q*±0.002 Å⁻¹, then the fibrous material of which the article is composed is determined to not exhibit a lamellar structure.

For a sample analyzed upon wetting from the dry, conditioned state, if an intensity peak is present at 2q*±0.002 Å⁻¹, the sample is determined to exhibit a lamellar structure, and the characteristic d-spacing parameter is defined as 2π/q*. If no intensity peak is present at 2q*±0.002 Å⁻¹, the sample is determined to not exhibit a lamellar structure. If a lamellar structure is determined to be present in at least any one of the three specimen/water ratios prepared, then the material of which the articles are composed is determined to exhibit a lamellar structure upon wetting. If no intensity peak is present at 2q*±0.002 Å⁻¹, in any of the three specimen/water ratios prepared, then the material of which the articles are composed is determined to not exhibit a lamellar structure upon wetting.

### Fibrous Element Composition Test Method

In order to prepare fibrous elements for fibrous element composition measurement, the fibrous elements must be conditioned by removing any coating compositions and/or materials present on the external surfaces of the fibrous elements that are removable. An example of a method for doing so is washing the fibrous elements 3 times with a suitable solvent that will remove the external coating while leaving the fibrous elements unaltered. The fibrous elements are then air dried at 23° C.±1.0° C. until the fibrous elements comprise less than 10% moisture. A chemical analysis of the conditioned fibrous elements is then completed to determine the compositional make-up of the fibrous elements with respect to the filament-forming materials and the active agents and the level of the filament-forming materials and active agents present in the fibrous elements.

The compositional make-up of the fibrous elements with respect to the filament-forming material and the active agents can also be determined by completing a cross-section analysis using TOF-SIMs or SEM. Still another method for determining compositional make-up of the fibrous elements uses a fluorescent dye as a marker. In addition, as always, a manufacturer of fibrous elements should know the compositions of their fibrous elements.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A water-insoluble fibrous element comprising:
a. one or more active agents;
b. one or more auxiliary ingredients; and
c. a fibrous element hydration controlling system.

2. The water-insoluble fibrous element according to claim 1 wherein at least one of the one or more active agents comprises a shave prep active agent.

3. The water-insoluble fibrous element according to claim 1 wherein at least one of the one or more auxiliary ingredients comprises a structurant.

4. The water-insoluble fibrous element according to claim 1 wherein at least one of the one or more auxiliary ingredients exhibits a weight average molecular weight of from about 10,000 to about 4,000,000 g/mol.

5. The water-insoluble fibrous element according to claim 1 wherein at least one of the one or more auxiliary ingredients is dispersed in the one or more active agents present in the water-insoluble fibrous element.

6. The water-insoluble fibrous element according to claim 1 wherein the water-insoluble fibrous element further comprises one or more optional active agents selected from the group consisting of: perfumes, colorants, preservatives, opacifiers, sunscreen agents, natural oils, vitamins, skin conditioning agents, skin soothing agents, sensates, powders, antioxidants, rosacea, skin benefit agents and mixtures thereof.

7. The water-insoluble fibrous element according to claim 1 wherein the fibrous element hydration controlling system comprises a C₈-C₂₂ quaternary ammonium compound.

8. The water-insoluble fibrous element according to claim 1 wherein the fibrous element hydration controlling system comprises a surfactant.

9. The water-insoluble fibrous element according to claim 1 wherein the water-insoluble fibrous element exhibits a lamellar structure response as measured according to the Lamellar Structure Test Method.

10. The water-insoluble fibrous element according to claim 1 wherein the water-insoluble fibrous element exhibits a lamellar structure response in a wet state but does not exhibit a lamellar structure response in a dry state as measured according to the Lamellar Structure Test Method.

11. A water-insoluble article comprising a plurality of water-insoluble fibrous elements according to claim 1.

12. The water-insoluble article according to claim 11 wherein the water-insoluble article further comprises an external fibrous element hydration controlling system.

13. The water-insoluble article according to claim 11 wherein the water-insoluble article exhibits a lamellar structure response as measured according to the Lamellar Structure Test Method.

14. The water-insoluble article according to claim 11 wherein the water-insoluble article exhibits a lamellar structure response in a wet state but does not exhibit a lamellar structure response in a dry state as measured according to the Lamellar Structure Test Method.

15. A method for making a plurality of water-insoluble fibrous elements, the method comprising the steps of:
a. providing a fibrous element-forming composition comprising one or more active agents, one or more auxiliary ingredients, and a fibrous element hydration controlling system; and
b. producing a plurality of water-insoluble fibrous elements from the fibrous element-forming composition.

16. A method for making a water-insoluble article, the method comprising the steps of:
a. mixing an external fibrous element hydration controlling system with a plurality of water-insoluble fibrous elements comprising one or more active agents and one or more auxiliary ingredients to form a mixture of the plurality of water-insoluble fibrous elements and the external fibrous element hydration controlling system; and
b. collecting the mixture of the plurality of water-insoluble fibrous elements and the external fibrous element hydration controlling system on a collection device to form a water-insoluble article comprising the plurality of water-insoluble fibrous elements and the external fibrous element hydration controlling system.

17. The method according to claim 16 wherein the plurality of water-insoluble fibrous elements are produced from a fibrous element-forming composition comprising the one or more active agents and the one or more auxiliary ingredients.

18. The method according to claim 17 wherein the plurality of water-insoluble fibrous elements are spun from the fibrous element-forming composition.

19. The method according to claim 16 wherein the water-insoluble article exhibits a lamellar structure response as measured according to the Lamellar Structure Test Method.

20. The method according to claim 16 wherein the water-insoluble article exhibits a lamellar structure response in a wet state but does not exhibit a lamellar structure response in a dry state as measured according to the Lamellar Structure Test Method.
